(19) United States
(12) Patent Application Publication
(10) Pub. No.: US 2003/0113813 A1
(43) Pub. Date: Jun. 19, 2003

(54) Methods and devices for the integrated discovery of cell culture environments

(76) Inventors: Mohammad A. Heidaran, Cary, NC (US); Mary K. Meyer, Durham, NC (US); Jon A. Rowley, Chapel Hill, NC (US); John J. Hemperly, Apex, NC (US)

Correspondence Address: BECTON, DICKINSON AND COMPANY, 1 BECTON DRIVE, FRANKLIN LAKES, NJ, 07417-1880, US

(21) Appl. No.: 10/260,737

(22) Filed: Sep. 30, 2002

Related U.S. Application Data
(60) Provisional application No. 60/335,898, filed on Nov. 15, 2001.

(51) Int. Cl. G01N 33/53; G01N 33/567; C12M 1/34; C12N 5/00

(52) U.S. Cl. 435/7.2; 435/287.2; 435/325

(57) ABSTRACT

The present invention is directed to methods and devices which can be used to test bioactive agents alone or in conjunction with 3D scaffolds for their effect on cell growth, differentiation, and other cellular functions.

### BACKGROUND OF THE INVENTION

1. Field of the Invention

The present invention in the field of cell biology, cell culture and tissue engineering is directed to methods and devices which can be used to test bioactive agents alone or in conjunction with three-dimensional (3D) scaffolds for their effects on cell growth and differentiation.

2. Description of the Background Art

The field of tissue engineering has emerged over the past decade due to a diverse range of clinical and non-clinical needs. These include replacement of diseased or damaged tissue and the delivery of genetically engineered cells to patients. The field has also been driven by a need for tissues which can be used in drug development where testing in animals is not always predictive of outcomes in humans.

The goal of tissue engineers is to meet these needs by creating living, three-dimensional tissues and organs using cells. In many cases, the approach is to coax cells into forming a tissue structure of the appropriate size and/or shape using a physical scaffold to organize cells on a macroscopic scale and provide molecular cues to stimulate appropriate cell growth, migration and differentiation. For example, in some applications, such as bone and blood vessel engineering, the donor material may be progenitor cells which can be stimulated to migrate, proliferate and differentiate, and then form appropriate tissue structures within a scaffold implanted into a site in the body.

In order for cells to properly grow, a scaffold must direct the arrangement of cells in an appropriate 3D configuration and present molecular signals in an appropriate spatial and temporal manner so that individual cells will form the desired tissue structures, and do so in a way that can be carried out reproducibly, economically and on a large scale. This goal requires that the scaffold comprise the appropriate biocompatible materials and bioactive agents to create an appropriate interaction between the scaffold and the cells.

The surface of a scaffold may have a large effect on cell traits, For example, texture, roughness, hydrophobicity, charge and chemical composition are surface properties known to affect cell adhesion to, and subsequent cell behavior on, a polymer surface. Thus, selection of a surface of paramount importance in the physical design of a scaffold.

Bioactive agents, such as the ligands present in the extracellular matrix (ECM), when deposited onto a scaffold surface, may act to enhance cell adhesion. ECM comprises a combination proteins, proteoglycans and charged polysaccharides which provides a physical scaffolding for cells and tissues. ECM helps provide a permeability barrier between tissue compartments and enables polarization of tissue structures.

Techniques of molecular and cell biology have been used to understand the structural/functional properties of many ECM molecules and map receptor-binding functions to small (3-20 amino acid) domains which are conserved among many different ligand proteins. The prototypical adhesion domain, the tripeptide, arginine-glycine-aspartate (RGD), was first identified as a minimal amino acid sequence of the ECM molecule fibronectin required for cell adhesion to fibronectin. RGD has since been found in a wider array of ECM molecules in which it mediates cell adhesion. Following the discovery of RGD, many such small adhesion-mediating peptide domains have been identified and characterized within ECM molecules. These peptides interact with a class of cell surface adhesion receptors called integrins which mediate many aspects of cell behavior. Therefore, the inclusion of peptide adhesion domains in synthetic biomaterials used to make scaffolds in order to manipulate integrin ligation can result in a more desirable cell and tissue culture environment.

Bioactive agents may not only effect the degree of adhesiveness of a cell culture surface, but may also induce physiological responses such as differentiation. For example, ECM molecules were included in a scaffold that enabled nerve cell differentiation. Culture of nerve cells on a scaffold comprising polyhydroxyethyl methacrylate did not result in ce1ll growth unless the ECM protein fibronectin was incorporated into the scaffold (Carbonetto, S. et al., Science 216: 897-899, (1982), herein incorporated by reference).

Another “class” of bioactive agents, growth factors, may act synergistically with ECM material or other agents to promote cell adhesion, differentiation or other cellular behaviors. Such agents may be incorporated into a scaffold or included in the culture medium. For example, morphological changes induced by recombinant growth and differentiation factor-5 (GDF-5) in fetal rat calvarial cells marked by cellular aggregation and nodule formation was dramatically enhanced by the presence of Type I collagen, but not fibronectin on the surface.

Moreover, this synergistic effect was highly specific to GDF-5 as compared to other mitogenic agents which failed to induce a similar response. This finding highlights the importance of identifying combinations of extrinsic factors required for optimal cell growth in vitro (Heidaran et al., E-biomed 2: 121-135, (2000) herein incorporated by reference).

Scaffold morphology can also have major effects on growth. Three-dimensional scaffolds may require a large surface-to-volume ratio within the 3D structures to support the adhesion of a large number of cells. Porosity of the scaffold material must be adequate to provide enough space for cells to penetrate the three-dimensional (3D) structure. Pores may be uniformly distributed and should be of appropriate size to permit adequate distribution of cells throughout the scaffolds.

Overall, then, design of an optimal in vitro environment for cells is a highly complex undertaking. The surface properties of a cell culture substrate, the contents of the culture medium, the choice of a two- or 3D environment and the interactions of these variable with any given type of cell being cultured are important components in determining success in achieving the desired outcome, generally cell growth. Yet, current methods used to optimize in vitro culture environments typically involve very limited testing of only growth or signaling factors that are added to culture medium while ignoring variables such as surface chemistry and morphology and interactions, sometimes synergistic, between such variables. Clearly, there is a need for new methods and devices that enable efficient and rapid assessment of the properties of cell growth surfaces, the components of the culture medium, synergy between growth and signaling factors as well as the “morphology” of the culture environment such as the impact of 3D scaffolds on cell growth and differentiation. Such a more complete analysis would enable a person skilled in the art to establish optimal in vitro environments for both the study of cell behavior and for practical applications including preparation of cell populations and cell-based biomedical devices for clinical use.

Cell-based assays for drug discovery are commonly performed using long-term or permanent, often genetically engineered, cell lines (see, for example, Housey, U.S. Pat. No. 4,980,281). Because such cell lines have typically been kept in culture for multiple generations and commonly originate from aneuploid tissues such as tumors, they do not represent ideal cells for study of normal physiological responses. Genetically altered cell lines may be unstable for numerous traits and thereby may not represent cells as they behave in vivo.

Many target and drug screening approaches are based on ligand binding assays. Use of such assays may identify as drug targets receptors or other binding molecules which are not physiologically or pathophysiologically relevant for the disease being targeted. Newer approaches to drug target identification include comparative gene expression profiling and certain proteomic methods in addition to target discovery based on conventional biology and cell signaling technologies. In traditional drug discovery, identification of a gene's (protein's) function and validation of its pathophysiological role are considered prerequisites to discovery of lead compounds.

The foregoing discovery processes are challenging, time-consuming and therefore often rate limiting, requiring extensive efforts to identify a target as being subject to the desired types of modulation by small molecule or macromolecular drug candidates. With the abundance of potential drugs in the post-genomic era, this traditional approach is not the most effective strategy. Rather, there exists a need, yet unmet, for methods that identify potential “druggable” targets in relevant model cell systems that best approximates the disease state in vivo. The present devices and methods are well-suited to meet these needs.

### SUMMARY OF THE INVENTION

The invention provides a device for assessing the effects of a 3D scaffold on a cellular activity, the device comprising a support surface having attached thereto a cell adhesion resistant (CAR) or low affinity material and a plurality of 3D scaffolds in operative contact with the surface. Also provided is a kit comprising this device.

Additionally, the invention includes a method for selecting components which when combined to form a culture system stimulate or promote or permit a desired cellular activity comprising the steps of (a) screening bioactive agents suitable for use as bioactive surfaces for the desired cellular activity; (b) screening a library of compounds for selection as suitable medium components or additives that support the desired cellular activity when the compounds are presented on or with the selected bioactive surface of (a); (c) fabricating a culture system comprising one or more 3D scaffolds comprising the selected bioactive surfaces of step (a) and the selected medium compounds of step (b); and d) determining whether the effects of the cell culture system achieve said desired cellular activity.

Further, the invention provides a method for designing an apparatus or device for assessing the effects of 3D scaffolds on cell activity comprising:

(a) screening bioactive agents for their suitability as components of a bioactive surface for promoting a desired cellular activity, which surface comprises a layer of a CAR material;

(b) depositing the bioactive agents onto or into a plurality of 3D scaffolds; and

(c) depositing the 3D scaffolds along with the associated bioactive agents onto a second support surface comprising said CAR layer.

The invention also includes an apparatus for screening bioactive surfaces comprising: a) a support surface having a layer of a cell adhesion resistant (CAR) material which is reacted with an oxidizing agent, the layer optionally bound to the support surface through an additional layer that comprises a polycationic polymer with amino groups such as polyethyleneimine (PEI), poly-L-lysine (PLL), poly-D-lysine (PDL), poly-L-ornithine (PLO), poly-D-ornithine (PDO), poly(vinylamine) (PVA), and poly(allylamine) (PAA), and c) a plurality of bioactive agents arrayed onto the layer.

One embodiment of the present invention is a cell culture device for assessing the effects of three-dimensional scaffolds on a desired cellular activity, comprising a culture vessel or support surface in contact with a layer of a cell adhesion resistant (CAR) material that forms a CAR surface, and a plurality of three dimensional scaffolds in contact with the CAR surface.

In this device, the CAR material may be bound irreversibly, generally namely, covalently to the surface. This is referred to herein as “bonding” or bonded”. The CAR material may also be bound reversibly to the surface, for example by electrostatic, ionic or hydrogen bonds, Van der Waals forces, hydrophilic/hydrophobic interactions, etc. Furthermore, the 3D scaffolds may be bound irreversibly or reversibly to the CAR material in the same manner.

Preferred CAR material is (a) polyethylene glycol, (b) glyme, (c) a glyme derivative, (d) poly-HEMA, (e) polyisopropylacrylamide, (f) hyaluronic acid (HA), (g) alginic acid (AA), (h) derivatives of HA or AA, and (i) a combination of any two or more of (a)-(h). Most preferred is HA.

The device may further comprise an additional layer comprising a reactive material between the support surface and the CAR layer. Preferred reactive materials for this layer are polyethyleneimine (PEI), poly-L-lysine (PLL), poly-D-lysine (PDL), poly-L-ornithine (PLO), poly-D-ornithine (PDO), poly(vinylamine) (PVA), and poly(allylamine) (PAA)

The CAR material is preferably susceptible to oxidation-mediated chemical activation.

The above device may further comprise a bioactive agent, such as an ECM material or a growth factor, present on or bonded to a surface of the scaffold. Examples of useful ECM molecules are vitronectin, fibronectin, tenascin, laminin, a collagen, entactin, a proteoglycan, aggrecan, decorin, biglycan, a glycosaminoglycan or Matrigel™. Examples of useful growth factor are a bone morphogenetic protein, epidermal growth factor, erythropoietin, heparin binding factor, hepatocyte growth factor, insulin, insulin-like growth factor I or II (IGF-I, II), an interleukin, a muscle morphogenic protein, nerve growth factor, platelet-derived growth factor or transforming growth factor α or β.

The scaffold of the device comprises a base material selected from the group consisting of (a) a natural polymer, (b) a synthetic polymer, (c) an inorganic composite and (d) any combination of (a)-(c).

The present invention provides a kit useful for optimizing a cell culture system cell growth and/or differentiation, comprising (a) any embodiment of the above device, and (b) packaging material. The kit of preferably further comprises (c) one or more reagents for use in cell culture and/or for measurement of the cell growth or differentiation, and/or (d) instructions for using the system.

The present invention is also directed to a method for designing a culture system that permits or promotes a predetermined amount or level of a cellular activity, and which system optionally enables assessment of the cellular activity, the method comprising;

(a) testing cells for the cellular activity after culture a culture vessel or on a culture surface in the presence of one or more first level combinations of

(i) a candidate culture medium component; and

(ii) a candidate bioactive surface comprising one or more bioactive agents presented to cells in or on one or more three dimensional scaffolds; which culture vessel surface and/or scaffold is in contact with a CAR material, thereby identifying an optimal first level combination of the medium components and the bioactive agents;

(b) selecting the optimal first level combination identified in step (a); and

(c) incorporating the selected optimal first level combination of medium components and bioactive agents into a culture system comprising a culture vessel or surface and one or more three-dimensional scaffolds have therein or thereon the selected optimal first level combination of medium components and bioactive surface.

This method may optionally further include a step of

(d) measuring the ability of the cell culture system to permit or promote the predetermined amount or level of the cellular activity.

The method may further comprise

(i) testing the cells for the desired activity after culture in the presence of one or more second level combinations of the medium components and the bioactive surface, thereby identifying an optimal second level combination;

(ii) selecting the optimal second level combination, and

(iii) incorporating the selected optimal second level combination of medium components and bioactive agents into a culture system comprising a culture vessel or surface and one or more three-dimensional scaffolds comprising the selected second level combination.

Also provided is a method for designing a culture system that permits or promotes a predetermined amount or level of a cellular activity, and which system optionally enables assessment of the cellular activity, the method comprising;

(a) testing cells for the activity after culture in a culture vessel or a culture surface, comprising a plurality of three dimensional scaffolds, in the presence of one or more combinations of

(i) one or more a bioactive agents bound to one or more CAR materials on the scaffolds, and

(ii) one or more culture medium components, thereby identifying an optimal combination of CAR material, bioactive agent and medium component;

(b) selecting the optimal combination identified in step (a),

(c) incorporating the selected optimal combination of CAR material, medium component and bioactive agent into a culture system comprising

(i) a culture vessel or surface comprising the CAR material of the combination,

(ii) in contact with the culture vessel or surface, one or more three-dimensional scaffolds having attached thereto the CAR material of the optimal combination thereby designing the culture system.

This method may further comprise:

(d) measuring the ability of the cell culture system to permit or promote the predetermined amount or level of the cellular activity.

The above measuring is preferably performed by cell imaging using an imaging apparatus operatively linked to the culture system. The invention therefore includes an apparatus comprising the culture system with its novel device(s) and an operatively linked imaging system. Any suitable imaging system that is commercially available or that may be designed is intended within the scope of the present apparatus, device and method.

In the foregoing method, the CAR material is preferably one of those noted above, and the bioactive agent, whether an ECM molecule, growth factor or otherwise, is preferably one or more of those described above.

In the foregoing method, the scaffold is preferably a material as described above.

The cellular activity measured in the above method is preferably growth or cell differentiation, either of which may be measured using an antibody-based assay.

This invention also provides a method for producing a device for assessing the effects, on a selected cellular activity, of a process of culturing cells in a culture system that comprises a three-dimensional scaffold, the method comprising:

(a) screening bioactive agents bound to a first CAR layer on a first support surface for their effect on the cellular activity by culturing cells in the presence of the agents, and selecting one or more of the agents that have a desired effect on the cellular activity;

(b) depositing the one or more bioactive agents selected in (a onto a three-dimensional scaffold to generate bioactive surfaces on the scaffold; and

(c) depositing a plurality of the scaffolds having the bioactive surfaces onto a second CAR support surface, wherein the first and the second support surfaces comprise either the same or different CAR material.

### BRIEF DESCRIPTION OF THE DRAWINGS

FIGS. 1A-1B depict cell growth on surfaces modified as described in Example 1.

FIG. 2 shows cells grown on the modified surfaces after fluorescent staining.

FIGS. 3A-3K shows murine MC3T3 cells adhering to and growing on several different concentrations of (FIGS. 3A-3E) and types of (FIGS. 3F-3K) ECM molecules.

FIGS. 4A-4F shows cell attachment and growth on a modified surface containing the ECM peptide, RGDSP (SEQ ID NO:1), in comparison to a control surface. FIG. 4A shows cells attached to circular regions in a grid on a petri dish. The immobilized HA surface was treated with increasing concentrations of NaIO₄ cross-wise (3, 10, 30 and 100 mM) or untreated (leftmost column “—NaIO₄”). The surface regions were then treated (top-to-bottom) with either buffer (1 μl) (bottom row) or equal volumes of various ECM materials, RGDSP—1 mg/ml; collagen—3.2 mg/ml; fibronectin—0.1% w/v; laminin—2 mg/ml; or a mixture termed of human ECM (hECM)—0.3 mg/ml. FIGS. 4B-4E shows enlarged micrographs of cells adhering to and growing on ECM vs. control surfaces as indicated.

FIGS. 5A-5B show the results of a medium optimization process wherein replacement of serum with defined growth factors enhances osteoblast growth (FIG. 4A) and differentiation (FIG. 5B). Murine MC3T3-E1 cells were studied.

FIGS. 6A-6C show covalently crosslinked 3D scaffolds arrayed on different plastic surfaces. A coin (U.S. 10 cent piece) is shown for comparison. FIG. 6C shows a plurality of scaffolds arrayed on a microscope slide.

### DESCRIPTION OF THE PREFERRED EMBODIMENTS

The invention provides devices for testing various bioactive agents and employing 3D scaffolds in to the design of an optimal cell culture environment. The invention also provides the methods of designing optimal cell culture environments.

In one embodiment, the device comprises a support surface to which is bound a cell adhesion resistant agent and to which are attached a plurality of 3D scaffolds.

Suitable support surfaces for use in the present invention include, but are not limited to ceramics, metals or polymers. The support surfaces may be in the form of dishes, flasks, microplates, tubes, sutures, membranes, films, bioreactors and microparticles, preferably made of a plastic. Preferred polymers for such surfaces include poly(hydroxyethylmethacrylate), poly(ethylene terephthalate), poly(tetrafluroethylene), poly(trifluoroethylene), poly(styrene) (“PS”), poly(vinyl chloride), poly(hexafluoropropylene), poly(vinylidine fluoride), poly(dimethyl siloxane) and other silicone rubbers. Glass support surfaces may include glycerol propylsilane bonded glass.

In one aspect of the invention, standard multiwell PS microplates (having 96, 384, or 1536 wells) are used, for example those manufactured by Becton Dickinson (Bedford, Mass.). In a alternate embodiment, 1536 well virtual, i.e., well-less, microplates of the type made by Becton Dickinson are used.

As used herein a “cell adhesion resistant” (or resistive) material, also referred to as a “low affinity agent,” is one which, when present on a surface, prevents or reduces cell adhesion or attachment. Such materials may also reduce protein binding. Suitable cell adhesion resistant agents include polyethylene glycol, glyme and derivatives thereof, poly-hema, poly-isopropylacrylamide and charged polysaccharides including, but not limited to, hyaluronic acid (HA) and alginic acid (AA). In general, highly hydrophilic substances with large numbers of hydroxyl groups may be used as cell adhesion resistant agents either alone or in combination. A preferred cell adhesion resistant agent is HA. Methods and compositions useful for creating CAR layers and CAR surfaces are described in greater detail in copending commonly assigned U.S. patent application Ser. No. ______, Liebmann-Vinson et al., “Cell Adhesion Resisting Surfaces” filed on even date herewith and hereby incorporated by reference in its entirety, as are the references cited therein.

A “reactive material” is one having groups such as amine or imine which can be deposited onto a support surface and to which the cell adhesion resistant agent may bond. Examples of such commercially available reactive materials include polycationic polymers, preferably polyethyleneimine (PEI), poly-L-lysine (PLL), poly-D-lysine (PDL), poly-L-ornithine (PLO), poly-D-ornithine (PDO), poly(vinylamine) (PVA), and poly(allyl amine) (PAA).

In a preferred embodiment, PEI is permitted to react ionically with a support surface. The PEI creates a surface of free amino groups which are available for coupling to a cell adhesion resistant agent using carbodiimide coupling (in one embodiment).

In another aspect of the invention, a cell adhesion resistant material is deposited onto a support surface which has been treated with an oxidative plasma. “Oxidative plasma treatment” includes exposure to plasma discharge in a vacuum or corona discharge. For plasma discharge in a vacuum, molded parts of a support surface are placed in a vacuum chamber and a mixture of gases including oxygen is pumped into the chamber. Under defined conditions of a partial vacuum and an electrical discharge, a reactive plasma, which reacts with the support surface is created. This process generates negatively charged functional groups on the surface including hydroxyl, carbonyl and carboxyl groups. Mixtures of other gases can also be added to create more complex oxidative plasma treated surfaces. For example, the surfaces of Primaria™ products (BD Biosciences, Bedford, Mass.) and CELL+™ products (Sarstedt, Newton, N.C.) contain both positively and negatively charged functional groups that can act to promote attachment of a cell adhesion resistant material.

Alternatively, after oxidative plasma treatment, a reactive material such as PEI may be deposited onto the support surface.

A “low affinity region”, also referred to as a “cell adhesion resistant: or CAR region is an area on a support surface onto which a cell adhesion resistant material has been placed, added, spotted, deposited, etc.

In one aspect of the invention, a plurality of 3D scaffolds are covalently or non-covalently immobilized to a low affinity region. In one embodiment, scaffolds are arrayed onto a HA-coated slide by injecting or otherwise placing the scaffolds into wells of a multi-well-slide having wells defined by a surrounding silicon gasket.

“Three-dimensional scaffold” refers herein to a 3D porous template that may be used for initial cell attachment and subsequent tissue formation either in vitro or in vivo. Three-dimensional scaffolds may comprise base materials (defined below) as well as other substances which may enhance cell growth, migration, adhesion and/or differentiation. Examples include peptides which promote cell adhesion.

Base materials for construction of these scaffolds include natural polymers, synthetic polymers, inorganic composites and combinations of these materials. Useful natural polymers include collagen-and glycosaminoglycans (GAG). One advantage of collagen as a base material is its natural abundance in tissues. The prevalence of collagen in the majority of human tissues underlies its ability to support the growth of a wide variety of cells and to support these tissues. Similarly, GAGs have physical and biological properties that make them desirable as tissue grafting base materials. In particular, GAGs control cell behavior and to play a role in tissue development and repair.

Synthetic polymers useful for scaffolding applications include poly(α-hydroxy acids) such as polylactic acid (PLA), polyglycolic acid (PGA) and copolymers thereof (PLGA), poly(orthoesters), polyurethanes, and hydrogels, such as polyhydroxyethyl methacrylate (poly-HEMA) or polyethylene oxide-polypropylene oxide copolymer (PEO-PPO). Poly(α-hydroxy acids) are among the few synthetic degradable polymers that are approved for human clinical use and have been used extensively for sutures.

Hybrid materials, containing natural and synthetic polymer materials, may also be used for 3D scaffolds of the present invention. Non-limiting examples of uses of these materials are disclosed, for example, in Chen, G. et al., Adv Materials 12:455-457 (2000), wherein hybrid sponges comprising collagen microsponges residing in pores of PLA or PLGA sponges were used to stimulate the proliferation and regeneration of a cartilaginous matrix from bovine articular chondrocytes. Carbonetto, S. et al., Science 216:897-899 (1982) used synthetic poly-HEMA hydrogel incorporated with either fibronectin, collagen, or nerve growth factor to stimulate growth of cultured neurons.

Inorganic composites are of special interest for bone substitute applications. In particular, calcium phosphate ceramics, bioglasses and bioactive glass-ceramics interact strongly and specifically with bone. Composites combining calcium hydroxyapatite and silicon stabilized tricalcium phosphate are another example of this class of materials.

Three Dimensional (3D) Scaffolds

It is understood that the 3D scaffolds of the present invention may comprise any of the base materials described above, singly or in combination as well as base materials known to those of skill in the art.

Three-dimensional scaffolds may be fabricated by well-known methods. A common fabrication process for both synthetic and natural scaffold materials involves is phase separation. In particular, phase separation upon freeze-drying has been used extensively (e.g., Zhang et al., J. Biomed. Mater. Res., 45:285-293 (1999); Ranucci et al., Tissue Engineering 5: 407-420, (1999); (Lu et al., Biomaterials 21: 1595-1605, (2000), each of which are herein incorporated by reference)). The base material is dissolved in a suitable solvent and rapidly frozen. The solvent is removed by freeze-drying leaving behind a porous structure. Non-limiting examples of scaffolds fabricated from natural polymers using this technique are porous collagen sponges with pores between about 50 and about 150 μm (Pieper et al., Biomaterials, 20:847-858 (1999) herein incorporated by reference), collagen-GAG scaffolds with an average pore size ranging from about 90 μm to about 120 μm (Hem et al., J. Biomed. Mater. Res. 39:266-276, (1998) herein incorporated by reference), chitosan hydrogels with pores ranging from about 45 μm to about 250 μm (Oxley et al. Biomaterials 14: 1064-1072, (1993) herein incorporated by reference)) and chitosan scaffolds with pore sizes ranging from about 1 μm to about 250 μm (Madihally, S. V. et al., Biomaterials. 21:1607-1619, (1999), herein incorporated by reference)) depending on freezing conditions. Examples for synthetic polymer scaffolds manufactured by freeze-drying are PLLA foams with porosity of up to about 95% with an anisotropic tubular morphology combined with an internal ladder-like structure containing channels ranging from several tens of micrometers to several hundred micrometers in diameter (Zhang et al., supra, 1999) and PLGA foams with about 90 to about 95% porosity and with average pore sizes ranging from about 15 μm to about 35 μm together with large pores of up to about 200 μm (Whang et al. Polymer 36:837-842, (1995), herein incorporated by reference). Freeze-drying has the added advantage that it allows incorporation of small hydrophilic or hydrophobic bioactive molecules into PLLA and poly(phosphoester) scaffolds as Thomson and coworkers have demonstrated (see, for example, Thomson et al., Polymer Scaffold Processing. In: Principles of Tissue Engineering, Lanza et al., eds., Landes Company, pp 263-272, (1997), herein incorporated by reference).

Modifications of the freeze-drying technique which are useful in making the present three-dimensional scaffolds are the “freeze-thaw technique” described by Oxley et al. (Biomaterials 14:1064-1072, (1993), herein incorporated by reference). This technique uses phase separation between a solvent, in particular water, and a hydrophilic monomer upon freezing, followed by polymerization of the hydrophilic monomer by UV irradiation and removal of the solvent by thawing. This leads to the formation of macroporous hydrogels. A second modification is freeze-immersion precipitation in which a polymer is dissolved in a solvent, cooled, immersed in a non-solvent, brought to room temperature followed by solvent removal, as demonstrated in the fabrication of polyester-urethane foams with pore sizes ranging from about 100 μn to about 150 μm (Saad, B. et al., J. Biomed. Res. 32:355-366, (1996), herein incorporated by reference). By combining phase separation with atomization and thin film deposition, PS foams with pore sizes up to about 100 μm have been fabricated (Gutsche, A. et al., Biomaterials 17:387-393, (1996), herein incorporated by reference).

Natural polymers can be formed into networks and gels suitable for 3D culture. For example, the polymerization of ECM proteins, such as fibrinogen, can be used to create networks with distances of about 250 nm and about 500 nm between fiber bundles (Herbert et al., J. Biomed. Mater. Res. 40: 551 (1998), herein incorporated by reference). Crosslinking of gelatin, a protein derived from collagen, and alginate create sponges with pores ranging from about 10 μm to about 100 μm (Choi et al., Biomaterials 20:409-417 (1999), herein incorporated by reference).

Three-dimensional printing is a fabrication technique similar to stereolithography. It involves selectively directing a solvent onto a polymer powder packed with NaCl particles to build complex 3D structures as a series of very thin two-dimensional slices followed by leaching of the NaCl particles in water. PLGA scaffolds with about 60% porosity and microspores ranging from about 45 μm to about 150 μm have been fabricated using this technology (Kim et al., Ann. Surgery 228:8-13, (1998), herein incorporated by reference).

Gas foaming methods involve the formation of a solid followed by exposure of this solid to a gas, e.g., CO₂, under high pressure which is allowed to saturate the polymer and after which the gas pressure is rapidly decreased. Pore formation occurs during pressure release due to the nucleation and expansion of the CO₂ dissolved in the polymer matrix. PLGA foams of porosity up to about 93% and with pore sizes of about 100 μm were prepared by this method (Mooney et al., Biomaterials 17:1417-1422, (1996), herein incorporated by reference)) reported the fabrication of PLGA foams with porosity of 97% and pore sizes ranging from about 10} μm to about 100 μm using this method.

Three-dimensional scaffolds may be fabricated by any known method including, but not limited to, those described above, or may be obtained commercially. Commercially available 3D scaffolds may be obtained from, for example, New Brunswick Scientific Co, Edison, N.J. (e.g., Fibra Cel®).

The 3D scaffolds used in the present invention may be of any suitable size or shape. In one embodiment, the 3D scaffolds are between about 1 to 50 mm in diameter. In another desired embodiment, the 3D scaffolds are between about 3 mm to 25 mm in diameter. In a yet additional desired embodiment, the 3D scaffolds are between about 3 mm to 10 mm in diameter.

The preferred properties of the surfaces of 3D scaffolds are those which permit cell adhesion and growth. Thus, any appropriate combination of texture, roughness, hydrophobicity, charge and chemical composition that meets this requirement is acceptable. In some cases, less adhesive surfaces that promote cell-to-cell rather than cell-to-surface adhesion may be preferred.

3D scaffold surfaces may incorporate a bioactive agent such as a peptide that permits or promotes cell adhesion, growth or differentiation. A “bioactive agent” or “bioactive component” or “bioactive compound” is one that affects physiological cellular processes, such as an agent that permits or promotes cell adhesion. For example, cell adhesion may be enhanced by any of a number of short peptides with sequences derived from adhesion proteins. These sequences bind to cell-surface receptors and mediate cell adhesion to the substratum with similar or lower affinity than the intact proteins. Arg-Gly-Asp-Ser-Pro (RGDSP) [SEQ ID NO:1] is one such peptide that may be coated onto the surfaces of 3D scaffolds to increase cell adhesion. This peptide binds to integrin receptors on a wide variety of cell types.

Preferably, the base material of the scaffold is one that does not support cell adhesion. Cell adhesive properties may be imparted to the scaffold by subsequent introduction of an adhesion protein or peptide such as those described above. For example, Han et al. (Macromolecules 30: 6077-6083 (1997), synthesized lactide-based PEG networks which showed cell adhesion resistance due to the surfactant PEG. When a cell adhesion resistant material is coated on the surface, adhesive properties can readily be introduced by introduction of an appropriate bioactive agent that bonds to the resistant material such as PEG through free terminal hydroxyl functions.

Other bioactive agents which bind to cell surface receptors and regulate the growth, replication or differentiation of cells include macromolecular growth factors (generally proteins), peptides, small molecules and extracellular matrix molecules, proteins (including modified proteins, e.g., glycoproteins), lipids, carbohydrates, nucleic acids, and the like.

Examples of growth factors are epidermal growth factor (EGF). platelet-derived growth factor (PDGF), transforming growth factors (TGFα, TGFβ), hepatocyte growth factor, heparin binding factor, insulin-like growth factor I or II, fibroblast growth factors, erythropoietin, nerve growth factor, bone morphogenic proteins, muscle morphogenic proteins, and other growth factors known to those of skill in the art. Other useful growth stimulating molecules include cytokines, such as the interleukins, and hormones, such as insulin. These are also described in the literature and are commercially available. See for example, Peptide Growth Factors and their Receptors” M. B. Sporn et al., eds., Springer-Verlag, New York 1990, which is herein incorporated by reference).

Growth factors can be isolated from tissue using conventional biochemical methods or produced by recombinant means in bacteria, yeast or mammalian cells (or other eukaryotic cells). For example, EGF can be isolated from the submaxillary glands of mice and TGF-β has been produced recombinantly (Genentech, S. San Francisco, Calif.). Many growth factors are also available commercially from vendors, such as Sigma Chemical Co. (St. Louis, Mo.), Collaborative Research (Los Altos, Calif.), Genzyme (Cambridge, Mass.), Boehringer (Germany), R&D Systems (Minneapolis, Minn.), and GIBCO (Grand Island, N.Y.), in both natural and recombinant form.

Examples of ECM molecules include fibronectin, laminin, collagens, and proteoglycans. ECM molecules are described in Kleinman et al., J. Biomater. Sci Polymer Ed, 1993, 5: 1-11 (herein incorporated by reference) or in other references are well-known to those skilled in the art. Many ECM molecules are commercially available. For example, a commonly used ECM material, Matrigel™, is made from the EHS mouse sarcoma tumor and is available from BD Biosciences, Bedford, Mass. Matrigel™ may contain laminin, collagen IV, entactin, heparin sulfate proteoglycan, growth factors, matrix metalloproteinases.

It is understood that the foregoing or other bioactive agents, whether growth factors, ECM molecules, or otherwise, may be incorporated into the 3D scaffolds according to the present invention.

In one embodiment, one or more bioactive agents, such as growth factor, are encapsulated in microspheres, preferably PLGA microspheres, and incorporated in that form into 3D scaffolds. (See, for example, Hile et al., J. Controlled Release 66 177-185, (2000), incorporated by reference). In another embodiment, one or more bioactive agents are dissolved in a carrier such as water to produce a solution that is used to coat the surface of 3D scaffolds. The latter is useful when growing anchorage-dependent cells. For example, a solution containing one or more bioactive agents is distributed onto or into 3D scaffolds and dried in a reverse airflow hood. This results in the deposition of the bioactive agents as a dried film on the scaffold surface. In yet another embodiment, a solution of one or more bioactive agents is prepared in 0.1M NaHCO₃ and shaken gently. A 3D scaffold is “coated” with this solution and is allowed to stand for about 2 hours at room temperature, after which the scaffold is washed with phosphate buffered saline (PBS) and seeded with cells.

In one embodiment, bioactive agents are immobilized onto three-dimensional scaffolds using well-known mild bioconjugation techniques such as those described in K. Mosback, Immobilized Enzymes and Cells, Part B, Academic Press (Orlando, Fla.), (1987); G. T. Hermanson et al., Immobilized Affinity Ligand Techniques, Academic Press, San Diego, (1992); and S. F. Karel et al., The Immobilization of Whole Cells: Engineering Principles, Chemical Eng. Sci. 40:1321 (1985)).

In one embodiment, the 3D scaffold comprises alginic acid (AA). One or more bioactive agents are coupled to the scaffold via the free carboxyl groups in AA using carbodiimide chemistry (preferably ethyldimethylaminopropyl carbodiimide or EDC) (J. Rowley et al., Biomaterials 20 45-53 (1999), herein incorporated by reference).

In another embodiment, A bioactive agent(s) is/are coupled to the scaffold using periodate chemistry. For this, the scaffold must comprise a polysaccharide that is partially oxidized by mild oxidants to convert some of the cis-diols to dialdehydes. These functional aldehyde groups can form Schiff's bases with free amine groups such as those present in bioactive polypeptides (N-terminal amino groups or side chain amino groups of Lys or Arg).

Preferably, a plurality, of 3D scaffolds comprising any of a number of base materials and/or bioactive agents are arrayed in a grid like pattern or a “microarray” on a low affinity layer that had been deposited onto a support surface. Each scaffold of the plurality may be identical.

The 3D scaffolds that comprise bioactive agents and which are arrayed onto a support surface, may be considered to act as a suitable “bioactive surface.” Such a bioactive surface present in and on the scaffolds preferably supports (a) the attachment and/or growth of a desired cell type, (b) the differentiation of cells to a desired cell type or (c) any other desired cellular activity. Thus, the term “bioactive surface” refers to a surface comprising a bioactive agent(s) which permit, promote, or enhance a desired cellular activity.

In one embodiment, cells are seeded into the 3D scaffolds of the device of this invention and are screened to determine which one or more of the plurality of 3D scaffold types (with respect to base material, deposited bioactive agent, etc.) results in expression of the desired cellular activity. Desired cellular activities include, but are not limited to altered growth patterns (e.g., enhanced or increased vs. inhibited or suppressed), altered cellular function, for example, transcription of a gene, translation of an mRNA, post-translational processing of a protein, intracellular transport, secretion or turnover of a protein or peptide. Such a protein may be an antigen, a toxin, an antibody, a hormone, a growth factor, a cytokine, a clotting factor, an enzymes, and the like. Altered activities of cells in culture also include changes in he synthesis processing and/or secretion of any endogenous or exogenous compounds or metabolites (e.g., an antibiotic, a steroid, a carbohydrate, a lipid, a nucleic acid, and the like). It is most preferred that the present devices and methods permit the induction and screening or identification of changes in the maturation, differentiation, growth and/or proliferation of cells.

The term “growth”, as used herein, means any increase in cell number (“proliferation”), cell size or in the quantity or concentration of a cellular component such as an organelle and/or an elongation of a cellular “process.” Cellular processes are extensions of the cytoplasm and may include specialized structures; examples include axons, dendrites, pseudopods, cilia, sensory endings, and flagella.

The term “differentiation” is well-known in the art, and as used herein, is intended to be broad, and thereby includes the potential of any and all types of stem or progenitor cells to produce more specialized or mature or committed progeny cells.

The term “differentiation” is well-known in the art is used here broadly to include the realization of the potential of any and all types of stem or progenitor cells to produce more specialized or mature or committed progeny cells.

The present invention is directed also to a method to screen for and discover a bioactive surface or surfaces which promote(s) a desired cellular activity. This method includes identifying optimal surface characteristics, which may, for example, subsequently be employed as a component of a 3D scaffold. In a preferred embodiment of this method, a suitable support surface as coated with a cell adhesion resistant material and optionally, with a reactive material (described above) and/or with a bioactive agent(s) deposited onto or bound to the cell adhesion resistant material. Cells are added and after appropriate culture intervals, are screened for the desired cellular activity.

In a preferred embodiment, a bioactive agent or agents are coupled to cell adhesion resistant or “low affinity” regions which may take the form of approximately circular spots, rectangular spots, ovoid spots, lines or any other suitable arbitrary shape. In a more preferred embodiment, bioactive agents are deposited onto a cell adhesion resistant surface in a “grid” or “microarray” pattern, i.e., arranged in a relatively uniform, spaced manner over an area described by two perpendicular axes.

The bioactive agents used in the present method are those described in connection with the present device. They may be covalently bonded to regions of a cell adhesion resistant surface to define a plurality of “bioactive regions.” Bioactive regions may differ from one another by either the nature or the concentration of the associated bioactive agent. Thus, in a preferred embodiment, each bioactive region comprises a different bioactive agent or combination of agents.

Once a cell adhesion resistant layer is formed on a surface support, the bioactive agent may be immobilized thereon using mild bioconjugation techniques known in the art, e.g., Mosback, supra;; Hermanson et al, supra; Karel et al., supra).

In a preferred embodiment, a bioactive agent is covalently coupled to a layer of HA. To do this, the HA is partially oxidized with a mild oxidant to convert some of the cis-diols to dialdehyde moieties. Mild oxidants include potassium permanganate and, more preferably, sodium periodate. The functional aldehyde groups thus created can form Schiff's bases with amino groups of bioactive agents, for example, ε-amino groups of Lys or Arg residues of a polypeptide or peptide.

The device of the present invention can be constructed of a support surface and an optional layer of a cell adhesion resistant material as described above, on which are deposited (e.g., covalently or non-covalently coupled) one or more bioactive agents to create a bioactive surface.

The deposited bioactive agents may then be screened for their effect on any desired cellular activity using any of a variety of methods described herein or known in the art.

This invention also provides a method testing a plurality of bioactive agents or different concentrations of an agent on a single cell culture surface for effects on cell growth and/or differentiation. The method includes forming a support surface having candidate bioactive agents thereon in the form of at least one CAR region and at least one bioactive region, and depositing cells onto each of these regions and determining the effects on cells that are in contact with each of said regions and thereby exposed to the bioactive molecules that constitute the bioactive region.

Related to the foregoing, the present invention is directed to articles, devices and methods in which different bioactive regions are juxtaposed on a single surface or in a single culture vessel, such that cells can respond to differences or gradients between regions. For example, juxtaposed bioactive regions may comprise different bioactive agents, or different concentrations of the same bioactive agent such that, if a concentration gradient of a particular bioactive agent can serve as an inducing signal for a cellular activity, for example, growth or differentiation, the cell can respond accordingly to such a gradient. Conditions such as these attained by the present invention would not occur in or on conventional culture surfaces or vessels when employing conventional cell culture methods. Thus, the present approach permits detection of signals and interactions that may be important in vivo but that are lost in the conventional cell/tissue culture environment.

In one embodiment of this invention, a bioactive region of a surface is compared with a CAR region of the same surface to determine if the bioactive agent inhibits growth and/or differentiation. In one embodiment, a bioactive agent acts as an inducing agent whereas a CAR material has no effect on cell differentiation. In another embodiment, an inducing agent or signal comes in the form of a gradient of one or more different bioactive regions that are juxtaposed on a single surface, such that cells can respond to the differences in bioactive agents or differences in concentration of a bioactive agent. Such induction is typically stimulatory to a cellular activity. However, in another embodiment a particular bioactive agent or a gradient created by juxtaposition of different bioactive regions acts to inhibit or prevent the induction of cell growth or differentiation (or other cellular activity).

In a preferred embodiment, a bioactive agent is coupled to one or more CAR regions in the form of a circular spot, a rectangular spot, an ovoid spot or a spot of any other arbitrary shape. Preferably, a bioactive agent is deposited onto a CAR material in a “grid pattern”, i.e., arranged as relatively uniformly spaced, horizontal and perpendicular spots.

The bioactive agent may be covalently bonded to a surface comprising a CAR material to create multiple bioactive regions each having a different concentration of the same agent. In another embodiment, each bioactive region comprises a different bioactive agent or a combinations thereof. Any combination of grids or other patterns wherein the same or multiple different bioactive agents are spotted is intended.

A cell or cells may be deposited onto a surface displaying CAR materials and/or bioactive agents. Although any cell type may be used in the present method, including prokaryotic and eukaryotic cells, most preferred are mammalian cells, particularly from humans, rats, mice or bovine species. In one preferred embodiment, stem cells are used.

In one embodiment of the present screening method, bioactive surfaces are screened in combination with medium components to identify synergistically acting combinations of bioactive agents and medium components. In another embodiment, medium components are first evaluated separately. Selected medium components with the desired effects on a particular cellular activity are then tested in combination with one or more bioactive agents in the form of bioactive regions (bioactive surfaces).

Peptide Libraries: Algorithms and Mathematical/Statistical Approaches to Screening

In a preferred embodiment, the screening and determination of desirable medium components is carried out employing the methods and software described in two commonly owned PCT patent publications, Campbell, R et al., WO 01/07642, and Haaland et al., WO 02/02591, both of which are herein incorporated by reference. As described in WO 01/07462, a desirable medium component, e.g., a bioactive agent the use of which results in a desired cellular activity is identified. A preferred class of bioactive agents for use as medium components are peptides or polypeptides.

In one preferred embodiment, a first test library of bioactive agents is evaluated to identify those library members of that have desired characteristics as components of a culture medium. A plurality of culture media, each containing a respective test compound(s) from the first test library are screened by measuring the effect of these media on a selected cellular activity. Typically, the culture media are screened as a plurality of first cell cultures each of which contains a respective culture medium including a respective test compound.

Compound libraries comprising bioactive agents can be generated by any method known in the art. Individual library members can be isolated and/or synthesized by solid phase or solution phase synthetic methods. For example, peptides can be synthesized by FMOC chemistry (Atherton et al., (1989) Solid Phase Synthesis: A Practical Approach. IRL Press at Oxford University Press, Oxford, England) on an Advanced ChemTech Model 396 synthesizer. Alternatively, peptides may be synthesized using other variations of the Merrifield approach (Merrifield, (1965) J Am. Chem. Soc. 85:2149), including t-Boc chemistry, synthesis on other solid supports (e.g., other resins, pins, etc.; “tea-bag” synthesis (R. A. Houghten (1985) Proc. Natl Acad. Sci. USA 82:5131), and by combinatorial methods. Peptides may also be modified at the carboxy terminus (e.g., esters, amides, etc.), the amino terminus (e.g., acetyl groups), and may include non-naturally occurring amino acids (e.g., norleucine). Methods of oligonucleotide synthesis are also known in the art. See, e.g., Oligonucleotide Synthesis: A Practical Approach, M. J. Gait, ed, IRL Press; Washington, D.C., 1984. The generation of carbohydrate libraries is described, e.g., in Liang et al. (1996) Science 274: 1520. Construction of RNA libraries are known in the art, e.g., by SELEX methods (C. Tuerck et al. (1990) Science 249:505.

In carrying out this embodiment, a first plurality of culture media each of which includes a first test compound or compounds is provided. Cell cultures are incubated under appropriate conditions for appropriate durations, know in the art, and assays are performed to determine qualitatively or quantitatively the occurrence of the targeted cellular activity.

The first test compounds are selected from a first test library of compounds, preferably using a space-filling design. The first test compounds should be representative of the first test library. The term “space filling design” as used herein is intended to be construed broadly and includes all such known techniques. Exemplary space-filling designs include but are not limited to full factorial designs, fractional factorial designs, maximum diversity libraries, genetic algorithms coverage design, spread design, cluster based designs, Latin Hypercube Sampling, and other optimal designs, e.g., D-Optimal and the like.

A space filing design assists in selecting experimental design points. Ideally, all data would be gathered at every possible combination of the explanatory variable which may possibly affect the response of interest, in other words, fill the entire space. When the candidate space is very large and the number of possible values is large, it many not be practical or efficient to enumerate all such possible combinations, much less physically gather the data. For example, it would generally not be feasible to evaluate all possible peptide tetramers or pentamers (i.e., 160,000 possible tetramers and 3,20,000 possible pentamers). Space filling designs provide a strategy for gathering data as a set for design points, such that the data gathered will efficiently represent all candidate compounds, known as the “candidate space.” One method of generating a space-filling design when no prior information is available is to use a geometric distance-based criterion.

Two general categories of distance-based designs are minimax and maximin. Assume that C denotes a finite set of possible design points and that there is a distance function d on C×C space such that (C,d) is a metric space. Consider subsets D of C of size n. D is called a distance-based design if the design criteria depends on the distance function d. The minimax criterion attempts to cover the experimental space by locating design points so as to minimize the maximum distance for a candidate point to the closest design point. More specifically, call D* a minimax distance design if

$${min}_{D} {max}_{y ∈ C} d ( y¸D ) = max d ( {y¸D}^{*} ) = {d}^{*}$$

where: d(y,D)=min_{x∈D} d(y,x)

The maximin criterion tries to spread the design points in space so as to maximize the minimum distance between the pairs of design points. In particular, Do is called a maximin distance design if

$${max}_{D } {min}_{x , {x}^{′} ∈ D} d ( x , {x}^{′} ) = {min}_{x , {x}^{′} ∈ {D}^{0}} d ( x , {x}^{′} ) = {d}^{0}$$

Maximin designs can be generated by Gosset (Hardin and Sloane (1992) Operating Manual for Gosset: A General Purpose Program for Constructing Experimental Designs (2d ed.), Mathematical Science Research Center, AT&T Bell Laboratories, Murray Hill, N.J.).

“Coverage” and “Spread” criteria are more numerically stable approximations to minimax and maximin criteria, respectively, that can be found [computed??] using an exchange algorithm. The maximin, or spread criterion, tends to produce designs with a large number of design points at the boundaries of the region or most extreme values, while the minimax, or coverage criterion, produces designs with more points in the interior of the region. A coverage design minimizes the following criterion for a choice of parameters p

and q:

$${C}_{p , q} ( C¸D ) = {\{ {\sum }_{y ∈ D} {( {d}_{p} ( y¸D ) )}^{q} / {N}_{C} \}}^{1 / q}$$

where the distance metric is defined as

$${{d}_{p} ( y¸D ) = {\sum }_{x ∈ D} {‖ x - y ‖}^{p} / n \}}^{1 / p}$$

where p<0 and q>0.

Alternatively, as another space-filling design, test libraries can be generated using a genetic algorithm which is generally based on the model of natural selection. Genetic algorithms optimize structures by computationally performing selection, crossover, and mutation in a population of structures in a manner analogous to natural selection. A given population of compounds is encoded as binary structures (“chromosomes”), and their opportunity to “reproduce” and to be included in succeeding generations is based on their biological activities. In the reproduction step, the chromosomes for two compounds are crossed at a single point to produce two new “child” compounds. Mutations occur by randomly changing any signal bit in the sequence. The chromosomes are then decoded into compound structures, which are then synthesized and tested. The process is repeated for the next generation.

A typical genetic algorithm runs as follows:

Step 1. Initialize a population of chromosomes, i.e., compounds. (This can be done randomly by a computer, or selected structures can be used to “seed” the initial populations.)

Step 2. Evaluate each chromosome in the initial populations (e.g., synthesize and test each peptide in the initial population).

Step 3. Create new chromosomes by mating current chromosomes; apply mutation and recombination as the parent chromosomes mate. (This is done by a program performing the mutation and recombination process on the indicia of the properties of the compounds which indicia have been fed into a computer.).

Step 4. Delete members of the population to make room for the new chromosomes. (The population is held to a fixed size).

Step 5. Evaluate the new chromosomes and insert them into the population.

Step 6. The process can end at this point, with the best chromosome(s) being selected; alternatively, additional generations can be followed by repeating Steps 3-5.

In selecting a peptide library using a genetic algorithm, the chromosomes may be individual peptides. Each amino acid may be represented as a binary string. For a 4-bit string, there are 16 possible combinations. If, for example, only 10 of the possible amino acids are used, 6 of these amino acids must be represented twice (e.g., Gly is represented by 1010 and 1011), so that all 16 possible combinations are assigned to an amino acid.

An initial population of tetrapeptides can be generated by a random number generator. Structures can be modified at this point because of synthetic complexities or to ensure that each amino acid is represented at each position, etc.

As an example, assume the following set of chromosomes (peptides) are generated:

If Gly-Ala-Leu-Gly is represented as the binary string 10100111000101010 and Ser-Ala-Pro-Val is represented as 0101011000110000 and a computer decided to “cross” them at their mid-points, two new children chromosomes/peptides would be generated and added to the population to test: Gly-Ala-Pro-Val and Ser-Ala-Leu-Gly.

Genetic algorithms are described in more detail, e.g., in J Sing et al., (1996) J. Amer. Chem. Soc. 118:1669, and Handbook of Genetic Algorithms, Lawrence David, ed., Van Nostrand Reinhold: New York, 1991.

The test compounds can be all of, or a subset of, the compounds in the first test library. The first test library can be selected on the basis of any known and desired criterion. For example, the first test library may include all possible pentapeptides (comprising naturally occurring and/or non-naturally occurring amino acids). Alternatively, the first library may contain all possible pentapeptides that utilize a set of ten selected amino acids. As another example, all of the compounds in the first test library may have a specific amino acid, a chemical class of amino acid or a subunit designated in a particular position(s). For example, the first amino acid may be set as (i) Ala or (ii) an aromatic amino acid.

One use of the present invention is the screening of a peptide library to identify member peptides that are effective when added as culture medium components. There is no a priori requirement of a peptide library that is used in this embodiment. Again, the amino acids of the library peptides may be naturally-occurring and/or non-natural synthetic amino acids. The library may also include D-amino acids or chemically modified or derivatized amino acids (e.g., phosphorylated, methylated, glycosylated, etc.). Moreover, the peptide library can be pre-defined to contain less than all of the possible naturally-occurring and/or synthetic amino acids. The peptide library may also be pre-defined as to the length of all the peptide members, e.g., same length or limited range of lengths. Alternatively, the library may be selected so that members vary in length, e.g., tetramers, pentamers and/or hexamers. Peptide libraries in which all of the members have the same length (e.g., 4, 5, 6, 7, 8, 9, 10, etc.) are preferred. In other embodiments, the peptide library is selected so that peptides have a length ranging between about four and about twenty residues. more preferably between about four and about ten residues.

In a preferred embodiment, one or more amino acid positions in the peptides is fixed (i.e., nonvariable) or limited to a specified amino acid(s) or class(es) of amino acids. For example, the residues at positions 4 and 5 might be fixed as a particular amino acid (e.g., Ala or Val) or class of amino acids (e.g., aromatic amino acids). Likewise, a library of 20-mers, can be designed such that only 5 of the positions may be variable with the other positions fixed based on any desired criterion, e.g., random assignment, prior chemical knowledge, ease of manufacture and/or cost of synthesis etc.

Those skilled in the art will appreciate that fixing or limiting the choice of amino acid(s) at a particular position or positions will reduce the total size of the library and may likewise decrease the time, expense, and/or technical difficulty of preparing and testing the library's peptides, identifying leads, and conducting further follow up studies, if necessary.

The present invention provides a method for screening a compound library (preferably a peptide library) in which at least one position is nonvariable or limited (e.g., for peptides, less than all possible amino acids). The first round of screening may yield a compound of interest (e.g., a culture medium component). Alternatively, leads are identified through repetitions of the process with successive screenings performed as described herein.

Presence or induction of a desired cellular activity is determined by performance of one or more relevant assay operations. In one approach, a range of cellular activities is used as the basis of establishing a test requirement. Culture medium samples in which components are varied are then tested in comparison with this activity range as an index. The test requirements may be determined at any stage in the process of identifying, for example, optimal culture medium components. The test requirement may be set a priori or, alternatively, it may be determined after a set or plurality of different culture media each containing a particular test compound, have been tested. Moreover, the test requirement may change during the screening process.

The test requirement may represent a threshold level or index of the desired property so that components being tested must meet or exceed this threshold (e.g., when screening for compounds that increase antibiotic production). Alternatively, the test requirement may represent a ceiling so that values falling below the test requirement may be the goal (e.g., when screening for compounds that suppress endotoxin production during drug fermentation processes). In yet another embodiment, the test requirement may relate to a range of values of a desired cellular activity, i.e., the test requirement may establish both a floor and a ceiling, which would permit the attainment of a balance between competing factors, e.g., cell growth vs. protein production). Those skilled in the art will appreciate that the test requirement may represent an optimal index or optimal indicia of the biological property taken alone (e.g., maximal immunogen production). Alternatively the test requirement may take into account other criteria such as feasibility, cost, time constraints, effects on other desired properties of, for example, the culture medium, etc.

In another embodiment, the test requirement may be qualitative, rather than quantitative, in nature. For example, if one is testing for the presence or absence of a particular response (i.e., a yes/no result). Those skilled in the art will recognize that for computational analysis of qualitative date, the qualitative data points are generally converted to quantitative values (e.g., response/no response may be scored as 1/0, respectively)

Operations are performed to determine a relationship between at least one parameter of the first test compounds and the measured cellular activity for each of the first test compounds. It is believed, and preferred, that a mathematical relationship, preferably a mathematical structure-cellular activity relationship between the parameter and the cellular activity of interest be obtained. There is no particular limit to the number of parameters used to determine the relationship. As a nonlimiting example, anywhere between two and ten, inclusive, are used.

Any known parameter (i.e., descriptor) that can be applied to characterize a compound may be used to carry out the present invention. Physical, chemical (including biochemical), biological and/or topological parameters may be employed to determine the relationship. The term “parameter” as used herein is also intended to encompass the principle components of S. Hellberg et al., (1987) J. Med Chem. 30:1126 (e.g., z1, z2, z3). The parameter(s) used to describe the test compounds can change in both number and type during the selection process. In addition, the parameter(s) can be a whole molecule parameters(s), sequence specific parameter(s) or a combination of both.

Illustrative parameters that may be employed according to the present invention include but are not limited to molecular weight, charge, isoelectric point, total dipole moment, isotropic surface area, electronic charge index, and hydrophobicity (e.g., as exemplified by measurements such as logP, HPLC retention times, other known methods of determining hydrophobicity) of the whole molecule or individual building blocks of the molecule (e.g., peptide, amino acid, nucleic acid, saccharide unit, etc.).

Calculations of parameters can be carried out by any known method, for example, using a computerized system, e.g., a Silicon Graphics computer or a PC. Total charge, molecular weight, and total dipole can be calculated using Sybyl 6.5 (Tripos). “Moriguchi logP” (designated MlogP or mlogP, a measure of hydrophobicity) can be calculated using a Sybyl Programming Language Script. Literature values of electronic charge index and isotropic surface area for amino acids are available, (see, e.g., E. R. Collantes et al. (1995) J. Med. Chem. 38:2705). A variation of electronic charge index can be prepared in an analogous manner using Gasteiger charges supplied by Sybyl instead of CNDO/2 charges used by Collantes et. al. (supra). Principal component descriptors z1, z2, and z3 are provided by Hellberg et al., supra. Calculations of the isoelectric point can be carried out using a Sybyl programming Language Script.

The relationship between the at least one parameter of the test compounds (medium components) and the measured indications of desired cellular activity for each of the test compounds is used to identify a second plurality of culture media. Each second culture medium contains a second compound(s) from a second test library. The second plurality of culture media are predicted to permit or promote a cellular activity that satisfies a test requirement. Typically, the second test compounds will be untested compounds although those skilled in the art will appreciate that one or more compounds from the first set of test compounds may be included in the second set of test compounds, e.g., as controls.

In particular embodiments, the second test library is chosen to include a subset of the total number of compounds that satisfy the test requirement. The second set of test compounds may include all of the test compounds in the second test library or alternatively a subset thereof. For example, the second test library may include all peptides having five amino acids that are predicted to permit or stimulate a certain level (i.e., the test requirement) of antibody production by cultured hybridoma cells when added to culture medium. Alternatively, and preferably, the second test compounds are selected from, and more preferably are representative of, the second test library. Even more preferably, the second test compounds are selected from the second test library using a space filling design as described above. Alternatively, the above-described process is carried out in an iterative fashion. A second relationship between at least on parameter of the second test compounds and the measured indicia will be determined, and a third set of test compounds from a third compound library is identified. As a further alternative, if the first test library yields a suitable compound, the screening process can end there without the need to generate a second test library or to engage in further compound screening.

The systematic methods described herein can be supplemented by knowledge of the chemical behavior in the process of selecting a follow-up library of compounds. For example, in screening a peptide library, it may become apparent that peptides containing a large aromatic residue exhibit a desired indication of a desired cellular activity. Accordingly, in a follow-up library, peptides having a large aromatic residue are enriched.

If a satisfactory peptide (i.e., one satisfying the test requirement) is not identified among the first set of test peptides, the screening process is repeated. A second set of untested peptides can then be selected by any means known in the art, and the parameters for the second set of peptides may be calculated.

As a further example of process optimization, a “cocktail” or mixture of compounds for use in formulating and optimizing a culture medium can be identified. A cocktail of compounds may give improved results over a single compound alone (i.e., demonstrating synergy). In addition, once a compound(s) to be used as a culture medium component has been identified, the medium's base formulation may be reformulated to include such a compound, thereby further improving the final medium.

The base medium may be reformulated at any point in the screening process. In some situations it is advantageous to formulate the initial base medium in a suboptimal manner with respect to a particular cellular activity so that the impact of a test compound on that activity is more readily observable. In the screening process, it may be advantageous to screen a compound in two different base media in parallel, either concurrently or sequentially: one medium is chemically “undefined” whereas the second medium is more chemically-defined culture.

The terms “chemically defined” and “chemically undefined” when referring to culture media are used herein according to their commonly accepted meanings in the art. In general, a “chemically defined” culture medium is one in which essentially all of the components therein are known and are present in known concentrations. A “chemically undefined” culture medium is one in which the identity and/or concentration of some medium component is unknown and only proportional values (such as total amino nitrogen) are known or accessible. Thus, any medium containing an undefined component is considered “undefined.” The term “semi-defined” is typically used in the art to describe a medium that contains a small proportion of undefined components. Yeast extracts and fetal calf serum are examples of undefined components commonly added to culture media.

During the process of reformulating the medium, it may be advantageous to avoid producing significant changes in the cell population in response to the new medium (i.e., to avoid subcloning the population). For example, when moving cultured cells from a complex culture medium containing protein hydrolysates to a more defined medium in which selected peptides are substituted for the protein hydrolysate, changes may occur in the cultured cell population. It may be desirable for the cultured cells to retain their ability to grow in the complex medium (with or without the added test compound(s)). The parallel screening process described above enable the investigator to maintain the characteristics of cells cultured in the reformulated medium. In addition, by parallel examination of cell viability and growth in undefined and defined media, it is possible to identify compounds that enhance performance in both types of media.

Thus, in one embodiment, a bioactive agent is screened for its effects on cellular activity in a chemically undefined medium, in a chemically defined medium, or both (in parallel).

Either a chemically defined or undefined culture medium may be used to screen or grow cells on a bioactive surface, whether in a two dimensional or 3D culture condition.

Cultured cells may be “conditioned” or “adapted” prior to screening with a bioactive agent added to the medium by “cycling” the cells at least once through their current growth medium and the base medium that will be used for the screening process. Typically, the current growth medium is an undefined or semi-defined medium, while the base medium for screening is chemically defined. This conditioning/adaptation process will increase the likelihood that cells will grow in both (the current medium which will become their former medium and the new base medium). Conditioning/adapting a cell line can enhance the reproducibility of results in growth assays and thereby increase assay resolution. In addition, as noted above, when the cells can grow in both a chemically defined and an undefined medium, it may be easier to identify desirable medium components.

As a further optional step, the cells may undergo one or two rounds of incubation in base medium alone that lacks the test compound(s)) prior to being exposed to the test compound(s). Subjecting the cells to such conditions has a two-fold effect. First, it prevents carry-over of undefined medium components from the previous culture medium to the new medium, thereby avoiding unintentional skewing of the screening results. Second, such transitional incubation enables the cells to adapt to the new basal medium, thereby increasing the probability that results obtained using the new medium reflect the effects of the test compounds added, not the basal medium itself.

The results obtained using the present methods (and those described in WO01/07642) do not necessarily lead to a fully defined culture medium. In other words, the final medium formulation discovered and optimized through the present screening process may include various undefined components, for example serum, protein hydroylsate, etc.

The inventive screening methods described herein (and in WO/07642) may be carried out using a base medium having no undefined protein components. Alternatively, the base medium may contain an undefined protein component(s) as long as the effects of a test compound are not confounded by the presence of the undefined protein component. In one embodiment, the base medium used in the screening process contains serum preferably less than about 30% (v/v), more preferably less than about 20% (v/v. Preferred serum concentrations in the base medium range from about 0.05% (v/v) to about 30% (v/v), more preferably 1% (v/v) to about 30% (v/v), still more preferably about 5% (v/v) to about 20% (v/v). Sources of sera include, but are not limited to, fetal bovine (calf) serum, adult bovine, horse serum, human serum (preferably of blood type AB) and the like.

In addition to an animal serum, other undefined protein components that are used in the medium include hydrolysates (e.g., enzymatic digests) such as tryptone, protease-peptone and the like, extracts (e.g., yeast extract) and infusions (e.g., organ or tissue infusions, such as brain-heart infusions), as those terms are understood in the art. The source of the undefined protein may be yeast, slaughterhouse offal, milk or other proteins (e.g., gelatin), or animal tissues or organs.

As herein defined, an “inducing agent” or “induction agent” is a substances which acts to promote a desired cellular activity such as growth or differentiation. A potential or known inducing agent may be further characterized herein as a “bioactive” agent. The bioactive surfaces and medium components described herein, as well as the materials comprising the 3D scaffolds or the very architecture of the 3D culture itself, or any combination thereof, may act as inducing agents on cultured cells. Thus, an inducing agent may be provided in the form of a surface or region of a cell culture material or vessels such that cell growth and/or differentiation may be promoted by the surface properties of the growth or contact surface which have been introduced to a support surface in the form of a cell adhesion resistant material and/or a bioactive agent which may be presented uniformly or in the form of discrete regions (spots, dots, microarrays) of the culture vessel surface.

Any of a large number of cell types may be used in the present devices and methods. One desired cell type is stem cells. “Stem cells” are defined here as cells that have the ability to divide continuously in culture while also giving rise to specialized, differentiated cells. They are undifferentiated or relatively undifferentiated, lacking the morphology or markers characteristic of mature or differentiated cells. Stem cells are generally characterized by their developmental or differentiative potential. Thus truly “totipotent stem cells” have the capacity to become, the embryo, extraembryonic membranes and tissues, and all postembryonic tissues and organs.

“Embryonic stem cells” (also referred to as ES cells or ESCs) are a type of uncommitted, totipotent stem cells isolated from embryonic tissue. When injected into embryos, ESCs can give rise to all somatic lineages as well as functional gametes. In the undifferentiated state ESCs are alkaline phosphatase-positive, express immunological markers characteristic of embryonic stem and embryonic germ cells, express telomerase and retain the capacity for extended self-renewal. Upon differentiation, ESCs become a wide variety of cell types, of ectodermal, mesodermal and endodermal origin. ESCs have been isolated from the blastocyst inner cell mass or from gonadal ridges of mouse, rabbit, rat, pig, sheep, primate, including human, embryos. (See for example, Thomson et al. Proc. Natl. A cad. Sci. USA 92 7844-7848 (1995), Thomson et al., Science 282:1145-1147 (1998); Shamblott et al., Proc. Natl. A cad. Sci. USA 95 13726-13731(1998), all of which are herein incorporated by reference.)

In normal embryonic development, totipotent cells proliferate and segregate into different lineages (lineage-commitment), forming the primary germ layers: ectoderm, mesoderm, and endoderm. Further progression of cells of these germ layers through progressive lineage-commitment into progenitor lineages further defines the differentiation pathways of the cells and their ultimate function.

While a majority of the cells in an organism are the result of cellular progression through the sequence of development and differentiation, a few cells appear to leave this pathway to become reserve stem cells that contribute to ongoing maintenance of a stem cell pool that can aid in “repair” of an organism as needed. Such cells are referred to here as adult stem cells (ASCs). ASCs include cells known as progenitor stem cells as well as pluripotent stem cells. “Progenitor stem cells” are those cell that are committed to a particular lineage (regardless of the inducing agent that may be added to the medium or substrate). As such, progenitor stem cells give rise to progeny limited of a single lineage within their respective germ layers, e.g., liver or thyroid (endodermal origin); muscle or bone (mesodermal origin.), neurons, melanocytes, epidermal cells (ectodermal origin). ASCs can remain quiescent and nonreplicating. In contrast, lineage-committed progenitor cells are capable of self-replication but have a limited life span before programmed senescence manifests itself.

While a majority of the cells in an organism are the result of cellular progression through the sequence of development and differentiation, a few cells appear to leave this pathway to become reserve stem cells that contribute to ongoing maintenance of a stem cell pool that can aid in “repair” of an organism as needed. Such cells are referred to here as “adult stem cells” (ASCs). ASCs include cells known as “progenitor stem cells” as well as “pluripotent stem cells.” “Progenitor stem cells” are those cells that are committed to a particular lineage and, as such, give rise to progeny of a single lineage within their respective germ layers, e.g., thyroid (endodermal origin); muscle, bone (mesodermal origin), neurons, melanocytes, epidermal cells (ectodermal origin). ASCs can remain quiescent and nonreplicating. In contrast, lineage-committed progenitor stem cells are capable of self-renewal but may have a limited life-span before programmed senescence manifests itself.

Progenitor stem cells can be further classified as multipotent, oligopotent or unipotent. As used herein, “multipotent progenitor cells” form multiple cell types within a lineage. “Unipotent progenitor cells” form cells of a single type. “Oligopotent stem cells” form cells of more than one type, but not all possible types, within a lineage.

To illustrate, the mature central nervous system (CNS) comprises three primary cell types: neurons, astroglia and oligodendroglia. Unipotent neural progenitor cells (NPCs) give rise solely (and invariably) to a single type of neuron or to astroglial cells or to oligodendroglial cells. Oligopotent NPCs can give rise to (a) neurons of a number of different neuronal phenotypes (e.g., sensory or motor neurons) but not to astroglia or (b) one type of neuron and one type of glial cell or (c) astroglia and oligodendroglia but not neurons. In contrast, a “multipotent” NPC generates progeny cells of all three CNS lineages.

“Pluripotent stem cells” are stem cells capable of giving rise to most tissues of an organism (derived from more than one embryonic germ layer). Pluripotent stem cells are not committed to any particular tissue lineage (“lineage-uncommitted). They can give rise to cells of

(a) endodermal origin including, but not limited to, the epithelial cells or their derivatives and/or parenchymal cells of the trachea, bronchi, lungs, gastrointestinal tract, liver, pancreas, urinary bladder, pharynx, thyroid, thymus, parathyroid glands, tympanic cavity, pharyngotympanic tube, tonsils, etc.;

(b) mesenchymal origin including but not limited to, skeletal muscle, smooth muscle, cardiac muscle, white fat, brown fat, connective tissue septae, loose areolar connective tissue, fibrous organ capsules, tendons, ligaments, dermis, bone, hyaline cartilage, elastic cartilage fibrocartilage, articular cartilage, growth plate cartilage, endothelial cells, meninges, periosteum, perichondrium, erythrocytes, lymphocytes, monocytes, macrophages, microglia, plasma cells, mast cells, dendritic cells, megakaryocytes, osteoclasts, chondroclasts, lymph nodes, tonsils, spleen, kidney, ureter, urinary bladder, heart, testes, ovaries, uterus, etc.;

(c) ectodermal origin including but not limited to neurons, oligodendrocytes, astrocytes, retina, pineal body, anterior pituitary, posterior pituitary, epidermis, hair, nails, sweat glands, salivary glands, sebaceous glands, mammary glands, tooth e, inner ear, ocular lens, etc.

Pluripotent cells can remain quiescent, and they can be stimulated to proliferate and are capable of extensive self-renewal while remaining lineage-uncommitted. Pluripotent cells can generate various lineage-committed progenitor cells from a single clone at any time during their life span. Lineage-commitment occurs under the influence of one or more inducting agents. Once induced to commit to a particular tissue lineage, pluripotent stem cells assume the characteristics of lineage-specific progenitor cells.

Non-limiting examples of progenitor cells include the unipotent myosatellite myoblasts of muscle; the unipotent adipoblast cells of adipose tissue, the unipotent chondrogenic cells and osteogenic cells of the perichondrium and periosteum, respectively. Oligopotent stem cells include the oligopotent adipofibroblasts of adipose tissue and the oligopotent adipofibroblasts of adipose tissue. A preferred type of pluripotent stem cells are the hematopoietic stem cells of bone marrow which are also found in peripheral blood an umbilical cord blood. Other pluripotent cells are pluripotent mesenchymal stem cells for the connective tissues.

The present invention may employ any type of stem cell or cell population derived therefrom, including a clonal population. The stem cells may be derived from any organism, though mammalian stem cells are preferred, including stem cells of humans and non-human primates as well as rodents, equines, canines or lagomorphs.

In the present methods, cellular activity can be assessed using any suitable means known in the art. Cellular activity is assessed when screening for a suitable bioactive agent and surface, suitable medium components, and when testing the synergistic effects of bioactive agents/surfaces medium components. In some embodiments, bioactive surfaces and media components are screened using the device of the invention, preferably in the presence of 3D scaffolds.

An important cellular activity that is screened for in the present invention is that of cell growth or proliferation. Numerous assays and approaches have been developed for assessing cell proliferation, including those based on staining followed by microscopic observation, turbidimetric methods, spectrophotometric methods (including colorimetry and measurement of light absorbance at a selected wavelength), direct counting of cells with an automated cell counter and/or automated plate counter, measurement of total cellular DNA and/or protein, impedance of an electrical field (e.g., Coulter counter), bioluminescence, production of carbon dioxide, oxygen consumption, or adenosine triphosphate (ATP) production, the like. Oxygen biosensors (e.g., by Becton Dickinson, Bedford, Mass.) may be used for certain measurements.

Differentiation may be assessed by expression analysis (e.g., mRNA expression), immunological analyses and histochemical analyses, or combinations thereof. For example, to determine whether a particular bioactive agent(s) alone or in conjunction with one or more specific media components and/or 3D scaffolds induces cell differentiation, one may analyze expression of nucleic acids corresponding to certain genes. Methods for such analysis are well known in the art and include amplification methods (e.g., polymerase chain reaction (PCR), strand displacement amplification, etc.) and hybridization of probe sequences such as in Northern analysis (see Sambrook et al.). Northern blots examine the expression of known (or unknown) genes. Alternatively, it is possible to generate cDNA libraries or to observe differential displays of genes that are expressed in stem cells or in cells derived from stem cells, before and after exposure to a test bioactive agent and medium components in conjunction with 3D scaffolds of the present invention. For example, Northern analysis was used to assess the presence of mRNA transcripts of myogenin and MyoD1 genes expression as a measure of the induction of myogenesis in a pluripotent stem cell clone. (See for example, WO01/2167, herein incorporated by reference.)

Additionally, assessment of the binding of an antibody to an antigen, the expression of which is characteristic of a cellular phenotype or differentiation stage, is used to assess differentiation. As used herein, an “antibody” is any immunoglobulin (Ig) molecule, or an antigen-binding (=epitope-binding) fragment thereof. The term encompasses polyclonal, monoclonal, and chimeric antibodies; the latter of which are described in U.S. Pat. Nos. 4,816,397 and 4,816,567.

The present method comprises examining a cell sample using an immunoassay that employs a detectably labeled antibody sufficient to recognize and bind to a stem cell, differentiated progeny cells or stem cells, or tissues that comprise such stem cells or progeny.

Methods for producing polyclonal antibodies are well known in the art. See, for example, U.S. Pat. No. 4,493,795 (Nestor et al.). A monoclonal antibody (mAb) or an Fab chain derived therefrom can be prepared using conventional methods and hybridoma technology See, for example, Harlow, E. et al. (eds), Antibodies—A Laboratory Manual (1988), and Harlow, E. et al. (eds), Using Antibodies: A Laboratory Manual (1998), both from Cold Spring Harbor Laboratory Press, New York, and which are incorporated herein by reference.

For example, in one embodiment, the presence of a differentiated cell expressing an epitope of interest will be detected by a detectably labeled primary antibody against that epitope or preferably, by an unlabeled primary antibody and a detectably labeled secondary antibody specific for the Ig isotype of the primary antibody. The presence of the detectably labeled antibody bound to the cell(s) is measured using any appropriate method that is specific for the particular type of label. This presence of bound antibody is indicative of differentiation having occurred. Use of a method that permits measurement of a bound antibody is also termed herein “visualization of the antibody”.

Antibody labels most commonly employed are radionuclides, enzymes, fluorescers which fluoresce when exposed to ultraviolet light, luminescers, and the like. Numerous suitable fluorescent agents useful as labels are known, including fluorescein, rhodamine, auramine, Texas Red, AMCA blue and Lucifer Yellow. Methods of conjugating these labels to proteins are routine. Preferred radionuclides are selected from the group consisting of ³H, ¹⁴C, ³²P, ³⁵S 36CI, ⁵¹Cr, ⁵⁷CO, ⁵⁹Fe, ¹²⁵I and ¹³¹I.

A particular detecting material is anti-rabbit antibody prepared in goats and conjugated with fluorescein through an isothiocyanate.

Enzyme labels are likewise useful and can be detected by any of the presently utilized colorimetric, spectrophotometric or fluorimetric techniques. The enzyme is conjugated to an antibody or a particle by reaction with bridging molecules such as carbodiimides, diisocyanates, glutaraldehyde and the like. Many enzymes are known for use in immunoassays, preferred examples being peroxidase, β-glucuronidase, urease and alkaline phosphatase.

Cells in culture can be treated with an antibody to a differentiation marker to determine if cell differentiation has occurred. Antibodies are known which identify cells as neurons, bone cells and the like. Non limiting examples of antibodies useful to detect markers indicative of particular cells types including the following: mesodermal markers indicative of muscle (e.g., myogenin [F5D, Developmental Studies Hybridoma Bank (DSHB)], sarcomeric myosin[MF-20 (DSHB)], fast skeletal muscle[MY-32, sigma] myosin heavy chain[A4.74], (DSHB), smooth muscle[smooth muscle alpha-actin, IA4 (Sigma)], cartilage (collagens type-II [CIICI, DSHB], bone (bone sialoprotein [WVIDI, DSHB], endothelial cells (endothelial cell surface marker [H-Endo, Accurate]); endodermal markers (α-fetoprotein [HAFP, Chemicon]) epithelial cell [HA4c19, DSHB]) and ectodermal markers (e.g., neural precursor cells [FORSE-I, DSHB], nestin [RAT-401, DSHB] neurons [8A2, DSHB]), neurofiliments [RT97, DSHB].

Histochemistry can be used to assess cell morphology and differentiation. Such characteristics include round central areas and spidery cell processes or long polygonal cells with intracellular fibers as an indicator of the neuronal phenotype; the presence of small rounded multinucleated or binucleated cells with a central nucleus and perinuclear vesicles as indicators of liver cells; the presence of multinucleated linear and branched structures indicate skeletal muscle; alkaline phosphatase activity may indicate bone differentiation. Calcium precipitation using silver nitrate may also be used to identify bone differentiation.

Once the preferred medium components and bioactive surfaces have been identified using the foregoing devices and methods, they may be applied to the fabrication of 3D scaffolds. Thus, scaffolds are made that have the preferred bioactive surface and their use in the methods of the invention enable the induction of the desired cellular activities in culture. In one embodiment, these 3D scaffolds are arrayed on a low affinity support surface to prevent the adherence of cells to the support surface in regions between the 3D scaffolds. This maximizes cell adherence to the scaffolds themselves, as is desired.

In one embodiment, cells are cultured on or in a 3D scaffold having a selected bioactive surface in the presence of culture medium that comprises selected medium components. In this embodiment, the 3D scaffold(s) is/are placed into a bioreactor and the cells are seeded in the three-dimensional scaffold and allowed to incubate. A “bioreactor” refers to any suitable container used for bioprocessing of cells to produce a desired tissue. A bioreactor may maintain an adequate supply of nutrients. Control of growth may be enhanced by inline monitoring and the adjustment of pH, oxygen, glucose and mechanical pressure within the bioreactor. A bioreactor comprising the 3D scaffolds having a selected bioactive surface is one embodiment of a device of this invention. Another embodiment of such a device further comprises and culture medium including the selected bioactive components. Another embodiment of this device further comprises cells that are to be cultured.

The methods and devices of this invention may be used in a high throughput format. Many high throughput culture systems and assay systems are known in the art, and typically include common components or devices. For example, according to this invention, cell dispensing and combinatorial media optimization may be accomplished using micro fluidic handling. Micro fluidics may accommodate and be compatible with cell processing and cell dispensing. Methods and apparatuses for precise handling of small liquid samples for biosample aspiration are described in U.S. Pat. No. 4,982,739), reagent storage and delivery is described in U.S. Pat. No. 5,301,797, and a partitioned microelectronic and fluidic device array for clinical diagnostics and chemical synthesis is disclosed in U.S. Pat. No. 5,585,069), just to name a few.

(0163) Standard 96, 384, 1536 polystyrene microplates, BDT 1536 virtual polystyrene microplates, standard polystyrene microscope slides and BDT polystyrene microslides are desirable support surfaces to use in the present invention.

An incubator may additionally be connected to a micro fluid handler which enables the support surfaces to be coated with low affinity and bioactive agents, cells introduced, and the cells imaged using an imager instrument to observe growth and differentiation.

Numerous methods are known for imaging fluorescent cells with a microscope and extracting information about the spatial distribution and temporal changes occurring in these cells. Many such methods and their applications are described in Taylor et al., Amer. Scientist 80:322-335, 1992. These methods have been designed and optimized for the preparation of small numbers of specimens for high spatial and temporal resolution imaging measurements of distribution, amount and biochemical environment of the fluorescent reporter molecules in the cells.

The ARRAYSCAN™ (Cellomics, Inc., U.S. Pat. No. 5,989,835), is an optical system for determining the distribution, environment, or activity of luminescently labeled reporter molecules on or in cells for the purpose of screening large numbers of compounds for effects on specific cellular activity. In using the ARRAYSCAN™ System, the user provides cells containing luminescent reporter molecules in an array of locations and scans numerous cells in each location, converting the optical information into digital data and utilizing the digital data to determine the distribution, environment or activity of the luminescently labeled reporter molecules in the cells. The ARRAYSCAN™ System includes the apparatus and computerized method for processing, displaying and storing the data. This system provides high content cell-based screening, as well as combined high throughput and high content cell-based screening using ?? a large microplate format. This apparatus and system may be used according to the present invention to detect differentiation.

An imager such as that described by Proffitt et al (Cytometry 24: 204-213, 1996) may be used in one embodiment of the invention. This is a semi-automated fluorescence digital imaging system for quantifying relative numbers in situ of cells pretreated with fluorescein diacetate (FDA). The system utilizes a variety of tissue culture plate formats, particularly 96-well microplates. The system consists of an epifluorescence inverted microscope with a motorized stage, video camera, image intensifier and a microcomputer with a PC Vision digitizer. TurboPascal™ software controls the stage and scans the plate, acquiring multiple images per well. The software calculates total quantity of fluorescence per well, provides for daily calibration, and configures for a variety of tissue culture plate formats. Thresholding of digital images and the use of vital fluorescent reagents (that fluoresce only in living cells) are used to reduce background fluorescence without removing excess fluorescent reagent.

This invention also provides a novel method and approach for drug target identification and validation. The method is rapid and efficient, enabling the testing of multiple factors of different classes to identify drug targets and ultimately drugs that are specific for the identified targets. Factors that can be used as “probes” in the present method include soluble factors such as proteins (e.g., various growth and survival factors, cytokines, morphogenetic proteins, death ligands), small molecules (e.g., peptides, steroids, other natural or synthetic organic chemicals); ions of inorganic salts (Ca⁺⁺, Zn⁺⁺). Another class of factors that influence cellular activity are ECM molecules which often act in vivo in an “insoluble” form because of the way they are found in organized tissues. Examples of ECM proteins have been discussed above and include molecules include (1) proteins such as vitronectin, fibronectin, tenacsins, laminin, collagen; (2) proteoglycans (PG) such as heparan sulfate proteoglycans (HSPG), aggrecan, decorin, biglycan, (3) glycosaminoglycans (GAG) such as hyaluronic acid (HA), chondroitin sulfate (CS), keratan sulfate (KS), heparan sulfate (HS) and dermatan sulfate (DS).

The present method can be used, for example with the general discovery methods described herein to determine in a high throughput and efficient system the functional effects of numerous combinations of environmental “factors” including growth factors, ECM molecules, and peptides, for example from peptide libraries that are tested as cell culture additives. The methods permit the use of products of genes with either known or unknown function in screening for targets.

The target discovery method can be viewed as being divided into the following steps:

(1) Obtain in a pairwise fashion, abnormal (e.g., tumor) cells, generally from diseased tissue of a subject, and “paired” normal cells of similar histologic origin;

(2) Expose cells of these two types in a systematic fashion culture environments that comprise a plurality of different factors selected as possible drug target candidates to obtain information (generally in the form of experimental/analytic data) reflecting selected cellular activity or function, for example, cell growth, differentiation, the concentration of one or more proteins or other biomolecules, activity of a protein (e.g., enzymatic activity), etc. The environment can be presented to the cells in various formats disclosed herein including 3D scaffolds and microarray formats.

(3) Analyze the data obtained above using a suitably sophisticated statistical technique (such as those described herein to identify a set or class of factors which have differential effects on a cellular activity of abnormal cells as compared to paired normal cells.

(4) Identify as targets one or more biochemical pathways, e.g., a metabolic or a signal transduction pathway, affected by this set or class of factors identified above, and optionally

(5) Validate the targets identified above by testing a known antagonist or agonist that acts in or on the selected pathway.

The types of cellular activities that may be measured in this systematic approach include, but are not limited to, growth and/or differentiation of cells in culture. Examples of responses are stimulation or inhibition of cell proliferation or cell differentiation, altered production or secretion of a cellular product which result from changes occurring at any level, including (1) transcription, (2) translation, (3) post-translational processing, (4) intracellular transport, (5) extracellular secretion, (5) turnover of peptides or proteins, (6) synthesis, processing and/or secretion of other non-proteinaceous molecules and metabolites from classes of compounds that include, carbohydrates, lipids, nucleic acids and steroids. Another type of response is turnover or metabolism of exogenous molecules such as antibiotics and xenobiotics.

Proteins which may selected as a measure of a preferred cellular activity include surface antigens, toxins, antibodies, hormones, growth factors, cytokines, clotting factors, enzymes and the like.

In one embodiment, the cell proliferation is the measured function. Cell proliferation can be assessed by any method known in the art, for example, by (1) staining and visual observation using (microscopy, (1) turbidity measurements, (2) spectrophotometric measurements (including colorimetry and measurement of absorbance of light at a selected wave length), counting with an automatic cell counter and/or automated plate counter, measurement of total cellular DNA and/or protein, impedance of an electrical field, bioluminescence, CO₂, oxygen, or ATP production or consumption and the like, or using an oxygen biosensor (Becton Dickinson, Bedford, Mass.).

Antibodies may also be used to assess differentiation. The term “antibody” is intended to include any immunoglobulin, and in some cases may refer to antibodies and fragments that bind a specific epitope. The term encompasses polyclonal, monoclonal, and chimeric antibodies, as will be familiar to those of skill in the art. Methods for producing polyclonal and monoclonal antibodies are familiar to those of skill in the art. Labels commonly employed for visualization of an antigen-antibody complex are radioactive elements, enzymes, chemicals that fluoresce when exposed to ultraviolet light, and others. A number of fluorescent materials suitable for use as labels are known, including fluorescein, rhodamine, auramine, Texas Red, AMCA blue and Lucifer Yellow. In one specific embodiment, anti-rabbit antibody prepared in goats and conjugated with fluorescein through an isothiocyanate is used. Antibodies may also be labeled with radioactive elements, such as, for example, ³H, ¹⁴C, ³⁵S, ³⁶Cl, ⁵¹Cr, ⁵⁷Co, ⁵⁹Fe, ¹²⁵I and ¹³¹I, and detected by standard counting methods.

Enzymes such as peroxidase, β-glucuronidase, urease and alkaline phosphatase may also be used to detect differentiation by labeling and detection with colorimetric spectrophotometric and fluorospectrophotometric methods.

A cell or cells is or are “abnormal” encompasses if they have altered characteristics or functions that result from (or result in) a disease state or a disorder. Any abnormal cells from a disease or disorder that can be isolated and prepared for culture and for which there are obtainable comparable or “paired” normal cells, can be used in the present methods.

Some classes and specific examples of diseases or disorders that can be addressed by the present invention are (a) neurological (e.g., Alzheimer's disease, Parkinson's disease, amyotrophic lateral sclerosis, multiple sclerosis, Huntington's disease; (b) metabolic diseases and disorders including diabetes mellitus, lipid storage diseases such as Gaucherie disease and Fabry's disease (c) benign and malignant tumors of any type, including sarcomas, carcinomas, melanomas, leukemias, lymphomas, myelomas, papillomas. The present method may be used to evaluate drug targets for solid tumors such as colon and colo-rectal carcinomas, ovarian cancer, breast cancer, prostate cancer, lung cancer and kidney tumors such as clear cell renal cell carcinoma or Wilms' tumor. Brain tumors including gliomas, astrocytomas and benign and malignant meningiomas, etc.,

In practicing the invention, a population of abnormal cells of interest are isolated (for example, from a solid tumor) along with comparable normal cells (for example, from normal surrounding tissue in the same subject, or comparable normal tissue from a different individual. These “paired” normal and abnormal cells are then cultured for a period to obtain separate more highly enriched or purified populations of “primary cells” that maintain the cell lineage and characteristics of the original abnormal and normal cells

These two populations of cells thus obtained are then exposed to a plurality of different factors in the formats described herein and at least one cellular response or activity is measured (e.g., cell proliferation), with the object of identifying a drug or chemical agent that shows differential effects on the normal and abnormal cell populations. This may be, for example, an agent that inhibits cell proliferation in the abnormal cells and either stimulates, or has no effect on proliferation in the normal cells, or an agent that induces differentiation in the abnormal cells but has no effect on the normal cells. When one or more such agents is identified, a biochemical pathway or receptor affected by the drug or chemical agent can be identified, suggesting a class or set of potentially effective drugs or chemical agents to be tested as potential pharmaceuticals. The biochemical pathway or receptor thus identified can be validated in further testing by comparing the effects of known agonists or antagonists of the pathway or receptor on the two populations. In this way, the invention provides a systematic, efficient and rapid method for identification of drug targets and new classes of pharmaceutical agents.

Having now generally described the invention, the same will be more readily understood through reference to the following examples which are provided by way of illustration, and are not intended to be limiting of the present invention, unless specified.

### EXAMPLE 1

### Coating Process Used in Discovery of Optimal Bioactive Agents for Use on a Surface

The BDT 1536 virtual plate (Becton Dickinson, Bedford, Mass.) used as the support surface in this example is a flat polystyrene sheet surface treated with a hydrophobic material to form virtual wells of exposed hydrophobic polystyrene on the same pitch and of the same diameter as standard 1536-well microplate. The BDT 1536 virtual plate also has the same dimensions as a standard microscope slide but is scored to form squares on the same pitch as a standard 96 or 384 well microplate.

Surface preparation and modifications is done using the following basic steps. (For more detail and schematic illustrations, see copending commonly assigned U.S. patent application Ser. No. ______, Liebmann-Vinson et al., “Cell Adhesion Resisting Surfaces” filed on even date herewith and hereby incorporated by reference in its entirety).

(1) Pre-treatment of surface, for example using oxidative plasma treatment or ammonia plasma treatment.

(2) optionally, treatment with a polycationic agent with free amino groups such as PEI, PLL, etc. (see above)

(3) coating with a CAR material, here HA

(4) mild oxidation of the CAR material with a mild oxidant, here sodium periodate

(5) Deposit of a bioactive agent and bonding to the oxidized HA by reductive amidation.

The slide is treated with oxidative plasma to activate the surface. Thereafter, PEI in aqueous solution is coated onto the slide and allowed to incubate for two hours. The slide is washed in water and left to dry. HA in buffer containing 1-ethyl-3-(3-dimethylaminopropyl)-carbodiimide (Pierce) (EDC) and N-hydroxysulfosuccinimide (Sulfo-NHS) which are added to provide reactive groups. EDC is a water-soluble derivative of carbodiimide that catalyzes the formation of amide bonds between carboxylic acids (or phosphates) and amines by activating carbonyl (or phosphate) to form an o-acylisourea-urea derivative. These groups are able to covalently bond compounds to the surface. Sulfo-NHS is water soluble and leaves a group for the activation of carboxylic acid.

Next, the slide is washed in buffer and left to dry for 1-2 hours. The slide is protected from light and oxidized with sodium periodate. After washing in buffer and drying, bioactive agents such as peptides are protected from light and coupled to the slide surface (though bonding of free amines to oxidized HA. The slide is washed with a mixture of water, salt and acetic acid, then washed with water and allowed to dry. The peptides are deposited in a grid or microarray pattern.

The bioactive agent such as an ECM molecule or a peptide in sodium acetate or other buffer is deposited after the oxidation of the HA. Viscosity of some bioactive agents in solution is evident at room temperature. Decreased viscosity is evident at 4° C. Pipetting is facilitated and stability is enhanced at room temperature or 4° C. Deposition of ECM molecules is preferably performed in the dark. Sterility is not required. Slides on which bioactive agents are deposited may be used within 1 hour.

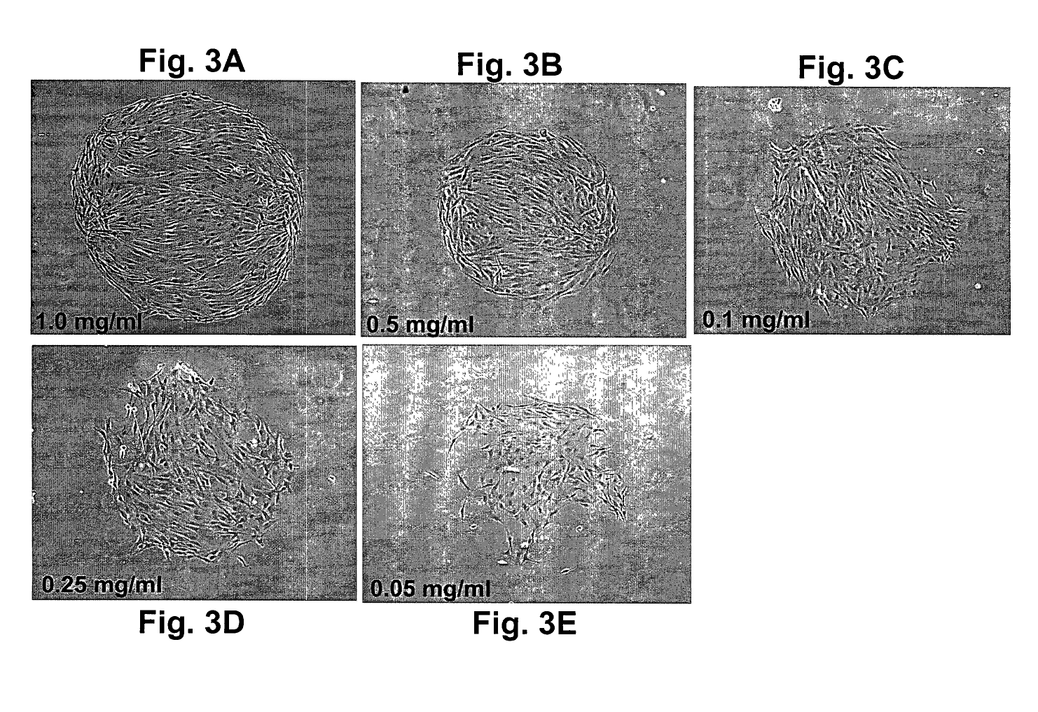

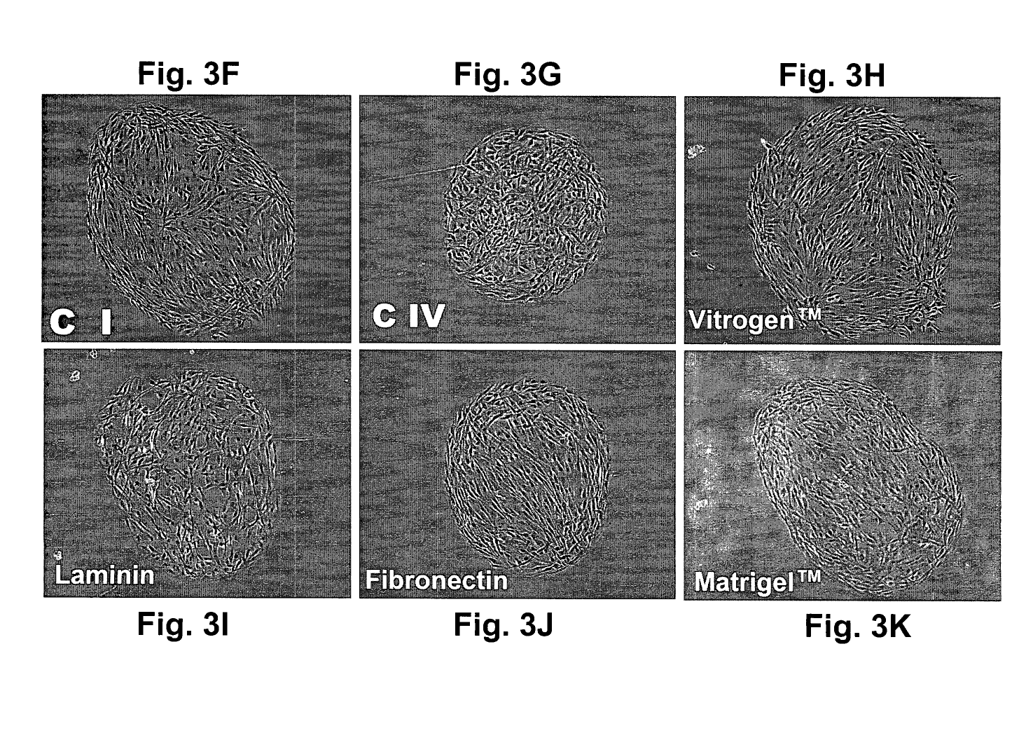

FIG. 3 depicts a microarray of bioactive agents coupled to a support surface modified as described. Varying concentrations of Vitrogen™ collagen, Collagen IV, fibronectin, MatriGel™, Collagen I, and laminin are coupled to a HA surface. Concentrations range from 1.0 to 0.05 mg/ml. Periodate or blanks are used as controls.

All ECMs are purchased from Becton Dickinson except for Vitrogen™ collagen which is obtained from Cohesion (Palo Alto, Calif., catalog number FXP-019) and fibronectin which is obtained from Sigma (St. Louis, Mo., catalog #F-0895). Additional ECMs included Type V Collagen, Laminin, Human Type I Collagen, Human ECM, Collagen Type IV, Collagen Type III, Human Collagen I. Polylysine is obtained from Becton Dickinson Labware and the peptide RGDSP (SEQ ID NO:1) is from AnaSpec Inc. (San Jose, Calif.).

### EXAMPLE 2

### Cell Growth on Modified Surfaces (from Example 1)

MC3T3-E1 cells were used to determine the effect of different bioactive agents for creation of bioactive surfaces influencing the growth and differentiation of these osteoblasts (also considered to be osteogenic stem cells). These cells are from a clonal line of murine calvaria-derived osteoblast (establishment of a clonal osteogenic cell line from newborn mouse calvaria describe in J Oral Biol 23:899 (1981)). The cells are maintained in α-MEM (GIBCO Catalog #12561) supplemented with 2% fetal calf serum (FCS; inactivated) and 100 units or μg/ml penicillin/streptomycin.

The cells were seeded at a concentration of approximately 10⁴ cells/20 ml culture medium in a 250 ml canted neck polystyrene tissue culture-treated flask. Cells were cultured until they reach about 2×10⁶ cells/flask. Cells were harvested and resuspended in 5 ml of α-MEM with 2% FCS to a final cell concentration of about 4×10⁴ cells/100 μl.

The surface modified slide was placed into a polystyrene Petri dish and covered with the cells for 30 minutes. Non-adherent cells were removed, revealing attachment only to the coated areas of the surface (See Figures). To quantitate cell attachment and growth, an image of the slide or other surface after staining with hematoxylin/eosin was captured using a color scanner, and the density determined using the Scion Image analysis program.

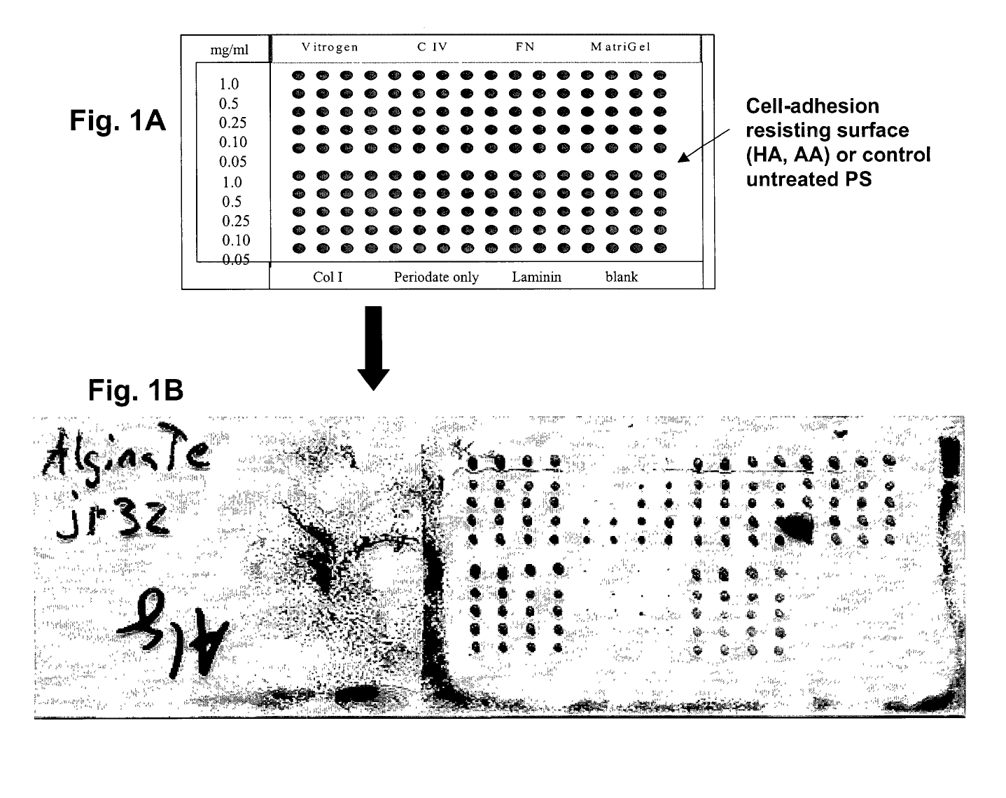

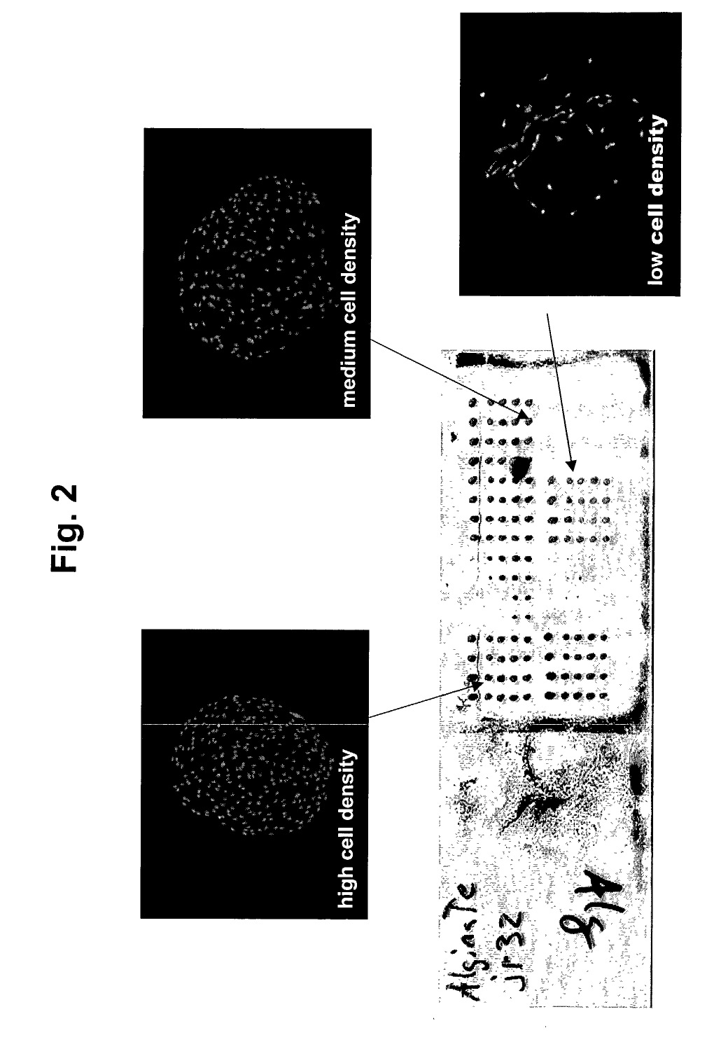

FIGS. 1A and 1B show that cells preferentially grew on particular areas of a microarray containing bioactive agents. Cells did not grow or adhere to the blanks or areas of the microarray that had been treated only with periodate. FIG. 2 shows that some cells grew less densely or more densely on particular bioactive agents. For example cell density was greater on Vitrogen™ than on laminin.

FIGS. 3A-3E shows that cell density is proportional to the concentration of the ECM molecule(s). Wells containing 1.0 mg/ml showed greater cell proliferation than did wells with surfaces coated with 0.05 mg/ml of the ECM molecules.

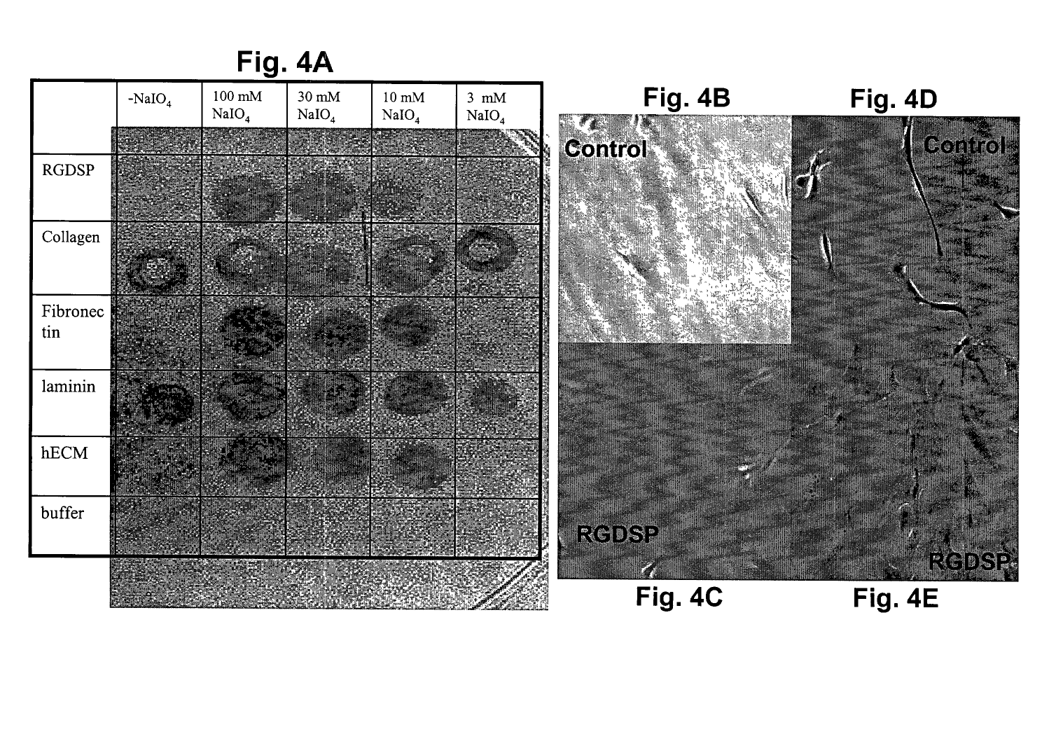

FIGS. 4A-4F show the effects of the modified surface on osteoblast cells. Surfaces not functionalized with bioactive agents do not promote cell proliferation. The three higher concentrations of periodate resulted in binding of all 5 ECM materials that stimulated cell attachment and growth. At the lowest (3 mM) periodate concentration, only collagen and laminin supported cell attachment and growth. In FIGS. 4B-4F, cells grew only on the surfaces onto which RGDSP was deposited an not on control surfaces.

### EXAMPLE 3

### Screening for Optimal Medium Components

Peptides which are tested in the optimization of medium components are from libraries that had been constructed using conventional techniques for peptide synthesis. Each library of initial peptide candidates is selected based according to specific design criteria including charge, molecular weight, mass and hydrophobicity. For example, each peptide of a library may consist of five amino acid residues corresponding to one of the following structures: (a) xxxKx [SEQ ID NO:2], (b) xxKxx [SEQ ID NO:3], (c) xxxxK [SEQ ID NO:4], (d) xKxxx [SEQ ID NO:5], and (e) Kxxxx [SEQ ID NO:6], wherein each x may be the same or different hydrophobic or uncharged polar amino acid residue and K is Lys. Peptides are synthesized with Lys at each of the five positions e with hydrophobic or uncharged polar amino acid residues present at the remaining four positions.

Peptides are synthesized by dispensing about 150 mg Wang Resin containing a desired first amino acid into a synthesis vessel. Next, the resin is swelled in 4 ml N-methylpyrolidinone (NMP) for 4 minutes with agitation. The first attached amino acid is deprotected twice with 1 ml of 20% w/v piperidine (Pip)/80% NMP for 20 minutes with agitation. This step is followed by washing with NMP (same duration and volumes as step 2) with agitation. The next amino acid is double coupled with 750 μl of amino acid stock at 2 molar excess, 500 μl of 0.5 M diisopropylcarbodiimide (DIC)/NMP and 250 μl of NMP for 60 minutes with agitation. Washing with NMP is repeated. Deprotection, washing with NMP, double coupling and washing again with NMP, is repeated for each additional amino acid. The resin then is washed with 10 ml of methanol over 10 minutes and then dried. The peptide is cleaved from the resin with 3 mls of 95% trifluroacetic acid (TFA)/5% water for 3 hours at room temperature. The resin is separated from the liberated peptide via filtration through glass wool. 80% of the TFA volume is removed. 4.5 mls of ether is added to the extract and then incubated for 0.5 hour at 4° C. overnight in a 10 ml vessel to enhance precipitation. The precipitate is pelleted by centrifugation for 20 minutes at 2200 rpm. Extraction, incubation and pelleting are repeated. The pellet is dried and 0.5 mls of acetic acid is added to the last pellet followed by 4.5 ml water. The pellets are frozen at −20° C. and lyophilized. 5 ml of water are then added and the pellets are frozen and lyophilized. Hydration, freezing and lyophilization are repeated and the processed peptide is maintained at −20° C.

Such a library of peptides may be tested to identify a peptide which when included in a culture medium will satisfy a test requirement (e.g., increased cell growth).

The following discussion is provided to illustrate the algorithm and approach described in greater detail in Campbell et al., WO 01/07642. In this example, eight test peptides are selected from a tetrapeptide library. The test peptides may be selected from the library by any known means. The values for three parameters (molecular weight, total charge, and hydrophobicity (mLogP) may be determined for each of the eight peptides. The indicia of the property, in this example, a particular cellular activity (i.e., growth) may be determined for the eight peptides as well.

Using regression analysis, e.g., with the program S-Plus (Version 3.4 for Solaris, Mathsoft, Seattle, Wash.), the following equation can be derived to describe the relationship between the three parameters and the (hypothetical) indicia of the property (i.e., growth) of the first set of test compounds.

ŷ=3.64×MlogP+0.056×MW−1.97×charge+1.73×R²=0.999 (1)

where ŷ is an estimated indicia of the property, MlogP is a measure of hydrophobicity, MW is molecular weight and R² is a statistical measure of the amount of variability in the original response variable (ŷ) that is explained by the statistical model. An R² value of 0.999 specifies that 99.9% of the original variability in ŷ was explained by the statistical model.

If a satisfactory peptide (that satisfies the test requirement) is not identified among the first set of test peptides, the screening process is continued. A second set of untested peptides can then be selected by any means known in the art, and the parameters for the second set of peptides may be calculated. Using Equation 1, the predicted activity of a second set of culture media, each of which includes one of the second group of test peptides, can be calculated for each culture medium in the second set based on the parameters of the peptide included therein. For example, a predicted activity of 28.2 may be derived for a culture medium containing an untested tetramer peptide. As this value is higher than any of the cellular activities in the original library, this peptide is classified as a good candidate for synthesis and testing.

If the growth requirement of the test is set at a level of at least 25 units, then the compound screening process may end with the identification of a specific tetrapeptide (assuming the actual cellular activity is equal to the predicted activity).

Alternatively, if the growth requirement had been set at least at 30, then the screening process would continue. The actual indicia of the property of a second set of culture media, each containing one of the second test peptides, may be determined. From these measurements, a new relationship between at least one parameter and cellular activity is calculated. From this updated equation, a third set of peptides, which when included in culture media are predicted to promote growth of the cells at a level of 30 or greater are identified. Typically, this process can continue in an iterative fashion until a peptide having the desired biological activity is identified.

Likewise, if the test requirement was set at a level of at least 20, then more of the original test peptides may satisfy the test requirement and the compound screening process could stop at this point or could continue in quest of even better performing peptides.

Referring to FIG. 2 of WO 01/07642, preferred operations for determining the relationship between the measured indicia of the property of the plurality of first culture media each containing a respective test compound and the parameter(s) of the test compounds can be illustrated by a graphical representation. The calculated values of the d parameter(s) are plotted, and the measured values of the indicia of the property for the n culture media are plotted against the parameter values in d+1 dimensional space. For ease of illustration only, in FIG. 2 of WO01/07642, n=10 culture media and d=1 parameter.

Conventional line-fitting algorithms can be used to generate a “best fit” line for the plotted data. For example, regression analysis can be utilized to determine a mathematical relationship between the indicia of the property and the value of the parameter for the test compound in each culture medium. The relationship can be represented as ŷᵢ=f(xᵢⱼ), where xᵢⱼ denotes a parameter, i ranges from 1 to n where n represents the number of first culture media, j ranges from 1 to d where d represents the number of parameters, and i represents an estimate of the measured first indicia of the property of the first culture media.

This relationship can be used to identify a second plurality of culture media each containing a respective second test compound which is predicted to provide indicia of the measured property that satisfies the test requirement. In FIG. 2 of WO 01/07642, the test requirement has been established to select for compounds that provide indicia of >20 units. The equation ŷ_{I}=f(xᵢⱼ) can be employed to identify those compounds as components of culture medium that will provide indicia of the property that lie on the upper right portion of the line of FIG. 2 of WO 01/07642 (i.e., that provide indicia of the property of >20 units).

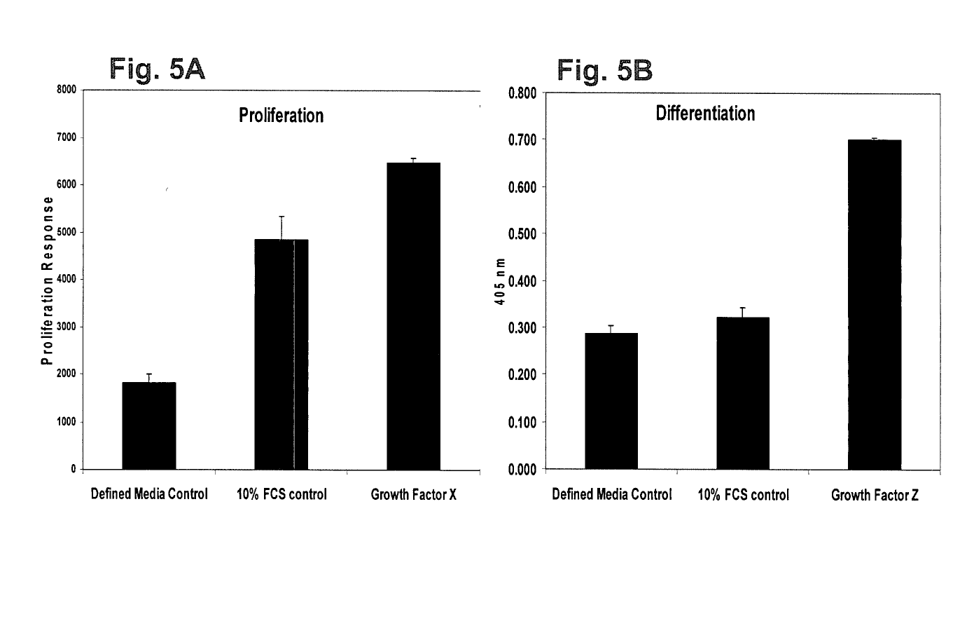

FIGS. 5A and 5B show the results of optimizing culture media for enhanced growth and differentiation of osteoblast cells. Proliferation and differentiation are improved when cells are grown on a bioactive surfaces in conjunction with an optimized media. Less proliferation and differentiation were observed using medium containing 10% FCS compared to media supplemented with growth factors in place of serum.

### EXAMPLE 4

### Use of Modified Surfaces as Three Dimensional Scaffolds

This example illustrates how the surfaces created as described in Example 1 and tested for a desired property such as enhanced cell growth as described in Example 2, may be used with 3D scaffolds to further enhance a desired cellular activity such as growth and/or differentiation.

After surface optimization and media optimization, the cells can be grown on a 3D scaffold comprising optimal surface components in the presence of an optimal growth medium.

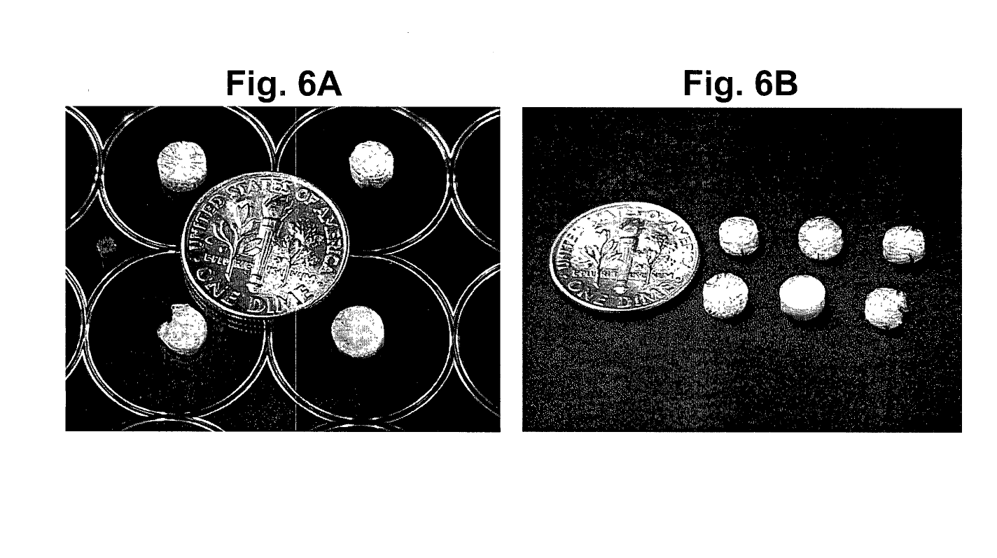

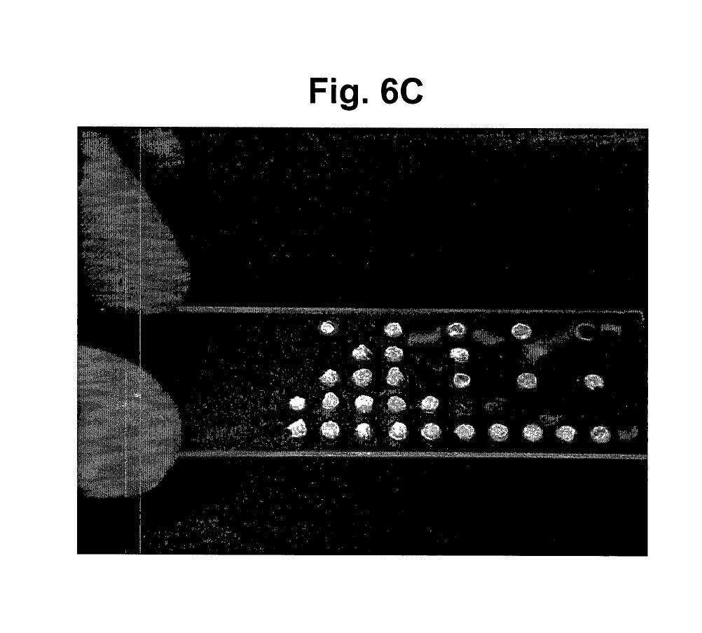

FIGS. 6A-6C show several 3D scaffolds of the present invention constructed using 3% alginate solutions in 0.1 M MES buffer, pH 6.0, which are crosslinked using EDC chemistry with a di-amine crosslinker adipic acid dihydrazide (AAD). The material is gelled between parallel PS surfaces with a 2 mm spaces to produce a homogeneous gel. A 6 mm hole punch is used to excise disks from a “sheet.” The disks are washed extensively in water, frozen at −100° C. and lyophilized until dry.

The 3D porous matrices have an interconnected pore structure which may have originated from the ice crystals. As the gels are frozen, ice crystals nucleate and grow. As the ice crystallizes, the polymer is excluded from the crystals. When lyophilized, the space occupied by the ice crystals may result in an interconnected pore structure. These matrices can be used for several purposes, including cell transplantation, in vitro 3D culture and screening of proteins and peptides for modulation of cell function.

The crosslinked alginates have available carboxyl groups for modification using EDC chemistry (See J. A. Rowley et al., Biomaterials (1999) 20:45-53, herein incorporated by reference), or using the same chemistry as described above for surface activation and coupling of bioactive agents. The 3D environment provided by the scaffolds mimics more accurately the in vivo environment, permitting use of cells growing in three dimensions almost as a tissue to screen growth environments, biological effects of drugs or other agents on cells and tissues in vitro.

The 3D alginate scaffolds are arrayed onto a HA-coated polystyrene slide as described in Example 1. See FIG. 6C. The 3D alginate scaffolds are arrayed by injecting the material into the wells of a 50-well silicone gasketed slide. The HA-coated slide is a preferred support for such scaffolds because cells that “spill out” of the scaffold during seeding will not adhere and grow on the surrounding surface while the scaffolds will be covalently bonded to the substrate via covalent crosslinks between the gelling scaffold and the polysaccharide (HA) substrate.

Arraying the matrices on a slide will allow automated screening of conditions optimal for development of in vitro engineered tissue. It is possible to seed different cell types on a variety of substrates at varying densities and/or co-culture cells of different types on the same scaffold to discover optimal conditions for engineered tissue development.

The crosslinked alginates described herein provide repeating carboxyl groups for modification according to Rowley et al., supra or using the chemistry described in Example 1. Thus alginates are modified with various biologically active agents including ECM molecules, growth factors, bioactive peptides, etc. The arrayed matrix slides used in conjunction with an automated imaging and analysis system allow for the automated discovery and optimization of environments (soluble factors, insoluble substances and 3D tissue compositions and architectures) useful for in vitro and even in vivo tissue development.

### EXAMPLE 5

### In vitro Assays for Monitoring Growth and Differentiation of Osteoblasts in Three Dimensional Scaffolds

Osteoblasts were grown in the 3D scaffold using an optimal medium that includes growth factors and an optimal surface. Low alkaline phosphatase (ALP) activity (detected calorimetrically using a commercial kit (from Sigma) according to the manufacturer's directions) and an excreted collagen matrix was evident between 1 and 9 days of culture. Between days 9 and 16, ALP activity accumulated and osteocalcin secretion occurred. ECM was mineralized after day 16. Greater cell proliferation, measured as oxygen consumption using the BD oxygen biosensor (Becton Dickinson, Bedford, Mass.) was observed in cells grown in a scaffold. The amount of DNA increased from day 0, day 3 and on day 10. ALP which also increased dramatically at Day 10, indicating increased differentiation. The ratio of ALP activity to DNA also increased on day 10.

All the references cited above are incorporated herein by reference in their entirety, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

What is claimed is:

1. A cell culture device for assessing the effects of three-dimensional (3D) scaffolds on a desired cellular activity, comprising a culture vessel or support surface in contact with a layer of a cell adhesion resistant (CAR) material that forms a CAR surface, and a plurality of 3D scaffolds in contact with said CAR surface.

2. The device of claim 1 wherein said CAR material is bound irreversibly to said surface.

3. The device of claim 1 wherein said CAR material is bound reversibly to said surface.

4. The device of claim 1 wherein said scaffolds are bound irreversibly to said CAR material.

5. The device of claim 1 wherein said scaffolds are bound reversibly to said CAR material.

6. The device of claim 1, wherein said cell adhesion resistant material is selected from the group consisting of (a) polyethylene glycol, (b) glyme, (c) a glyme derivative, (d) poly-HEMA, (e) polyisopropylacrylamide, (f) hyaluronic acid, (g) alginic acid, (h) a derivative of hyaluronic acid or alginic acid, and (i) a combination of any two or more of (a)-(h).

7. The device of claim 4, wherein said cell adhesion resistant material is hyaluronic acid.

8. The device of claim 1 further comprising an additional layer comprising a reactive material between said support surface and said CAR layer.

9. The device of claim 8, wherein said reactive material is polyethyleneimine, poly-L-lysine, poly-D-lysine, poly-L-ornithine and poly-D-ornithine.

10. The device of claim 1, wherein said bonded CAR material is susceptible to oxidation-mediated chemical activation.

11. The device of claim 1, further comprising a bioactive agent present on or bonded to a surface of said scaffold.

12. The device of claim 11, wherein said bioactive agent comprises an extracellular matrix molecule or a growth factor.

13. The device of claim 12, wherein said extracellular matrix molecule is vitronectin, fibronectin, tenascin, laminin, a collagen, entactin, a proteoglycan, aggrecan, decorin, biglycan, a glycosaminoglycan or Matrigel™.

14. The device of claim 13, wherein said extracellular matrix molecule is a collagen.

15. The device of claim 12, wherein said bioactive agent is a growth factor.

16. The device of claim 15, wherein said growth factor is a bone morphogenetic protein, epidermal growth factor, erythropoietin, heparin binding factor, hepatocyte growth factor, insulin, insulin-like growth factor I or II (IGF-I, II), an interleukin, a muscle morphogenic protein, nerve growth factor, platelet-derived growth factor or transforming growth factor α or β.

17. The device of claim 1, wherein said scaffold comprises a base material selected from the group consisting of (a) a natural polymer, (b) a synthetic polymer, (c) an inorganic composite and (d) any combination of (a)-(c).

18. A kit useful for optimizing a cell culture system cell growth and/or differentiation, comprising
(a) the device of any of claims 1-17, and
(b) packaging material.

19. The kit of claim 18 further comprising:
(c) one or more reagents for use in cell culture and/or for measurement of said cell growth or differentiation.

20. The kit of claim 18 further comprising
(d) instructions for using said system.

21. The kit of claim 19 further comprising
(d) instructions for using said system.

22. A method for designing a culture system that permits or promotes a predetermined amount or level of a cellular activity, and which system optionally enables assessment of said cellular activity, said method comprising;
(a) testing cells for said cellular activity after culture a culture vessel or on a culture surface in the presence of one or more first level combinations of
(i) a candidate culture medium component; and
(ii) a candidate bioactive surface comprising one or more bioactive agents presented to cells in or on one or more 3D scaffolds;
which culture vessel surface and/or scaffold is in contact with a CAR material,
thereby identifying an optimal first level combination of said medium components and said bioactive agents;
(b) selecting said optimal first level combination identified in step (a); and
(c) incorporating said selected optimal first level combination of medium components and bioactive agents into a culture system comprising a culture vessel or surface and one or more 3D scaffolds have therein or thereon said selected optimal first level combination of medium components and bioactive surface,
thereby designing said culture system.

23. The method of claim 22, further comprising:
(d) measuring the ability of said cell culture system to permit or promote said predetermined amount or level of said cellular activity.

24. The method of claim 22, further comprising
(i) testing said cells for said desired activity after culture in the presence of one or more second level combinations of said medium components and said bioactive surface, thereby identifying an optimal second level combination;
(ii) selecting the optimal second level combination, and
(iii) incorporating said selected optimal second level combination of medium components and bioactive agents into a culture system comprising a culture vessel or surface and one or more 3D scaffolds comprising said selected second level combination.

25. A method for designing a culture system that permits or promotes a predetermined amount or level of a cellular activity, and which system optionally enables assessment of said cellular activity, said method comprising;
(a) testing cells for said activity after culture in a culture vessel or a culture surface, comprising a plurality of 3D scaffolds, in the presence of one or more combinations of
(i) one or more a bioactive agents bound to one or more CAR materials on said scaffolds, and
(ii) one or more culture medium components,
thereby identifying an optimal combination of CAR material, bioactive agent and medium component;
(b) selecting said optimal combination identified in step (a),
(c) incorporating said selected optimal combination of CAR material, medium component and bioactive agent into a culture system comprising
(i) a culture vessel or surface comprising the CAR material of said combination,
(ii) in contact with said culture vessel or surface, one or more 3D scaffolds having attached thereto the CAR material of said optimal combination
thereby designing said culture system.

26. The method of claim 25 further comprising:
(d) measuring the ability of said cell culture system to permit or promote said predetermined amount or level of said cellular activity.

27. The method of claim 25 wherein said measuring is performed by cell imaging using an imaging apparatus operatively linked to said culture system.

28. The method of claim 25, wherein said CAR material is selected from the group consisting of (a) polyethylene glycol, (b) glyme, (c) a glyme derivative, (d) poly-HEMA, (e) poly-isopropylacrylamide, (f) hyaluronic acid, (g) alginic acid, (h) a derivative of hyaluronic acid or alginic acid, and (i) a combination of any two or more of (a)-(h).

29. The method of claim 28 wherein said CAR material is hyaluronic acid.

30. The method of claim 22 or 25, wherein said bioactive agent comprises an extracellular matrix molecule or a growth factor.

31. The method of claim 30, wherein said extracellular matrix molecule is vitronectin, fibronectin, tenascin, laminin, a collagen, entactin, a proteoglycan, aggrecan, decorin, biglycan, a glycosaminoglycan or Matrigel™.

32. The method of claim 31, wherein said collagen is collagen VI.

33. The method of claim 22, wherein said surface is treated with an oxidative plasma prior to creation of said CAR layer.

34. The method of claim 22, further comprising an additional layer comprising a reactive material between said vessel or surface and said CAR layer.

35. The method of claim 34, wherein said reactive material comprises polyethyleneimine, poly-L-lysine, poly-L-lysine, poly-L-ornithine, poly-D-ornithine.

36. The method of any of claims 22 wherein said CAR layer is treated with an oxidizing agent.

37. The method of claim 22 wherein said bioactive agents are deposited onto said CAR layer as a microarray.

38. The method of claim 30 wherein said bioactive agent is a growth factor.

39. The method of claim 22, wherein said scaffold comprises a base material selected from the group consisting of (a) a natural polymer, (b) a synthetic polymer, (c) an inorganic composite and (d) any combination of (a)-(d).

40. The method of claim 39, wherein said scaffold comprises collagen.

41. The method of claim 22 wherein said cellular activity is cell growth or cell differentiation.

42. The method of claim 41 wherein said cell growth or differentiation is measured using a detectably labeled antibody.

43. A method for producing a device for assessing the effects, on a selected cellular activity, of culturing cells in a culture system that comprises a 3D scaffold, said method comprising:
(a) screening bioactive agents bound to a first CAR layer on a first support surface for their effect on said cellular activity by culturing cells in the presence of said agents, and selecting one or more of said agents that have a desired effect on said cellular activity;
(b) depositing said one or more bioactive agents selected in (a onto a 3D scaffold to generate bioactive surfaces on said scaffold; and
(c) depositing a plurality of said scaffolds having said bioactive surfaces onto a second CAR support surface, wherein said first and said second support surfaces comprise either the same or different CAR material.
